(19) United States
(12) Patent Application Publication
(10) Pub. No.: US 2005/0130245 A1
(43) Pub. Date: Jun. 16, 2005

(54) Diagnosis and treatment of early pre-type-1 diabetes utilizing neuronal proteins

(75) Inventors: Jean-Francois Houle, Toronto (CA); Peter Kupchak, Toronto (CA); George Jackowski, Kettleby (CA); Anthony I. Kahama, Brampton (CA)

Correspondence Address: MCHALE & SLAVIN, P.A., 2855 PGA BLVD, PALM BEACH GARDENS, FL, 33410, US

(73) Assignee: Syn X Pharma, Inc., Toronto (CA)

(21) Appl. No.: 10/950,221

(22) Filed: Sep. 24, 2004

Related U.S. Application Data
(63) Continuation-in-part of application No. 09/954,972, filed on Sep. 17, 2001.

(51) Int. Cl. G01N 33/53; G01N 33/537; G01N 33/543

(52) U.S. Cl. 435/7.92

(57) ABSTRACT

This invention relates to the diagnosis and treatment of pre-Type 1 diabetes and Type-1 diabetes (T1D); particularly to the use of neuronal proteins as predictors of the disease; and most particularly to GFAP (glial fibrillary acidic protein); GAD65 (glutamic acid decarboxylase 65); NSE (neuron specific enolase; S100β and CNPase (2′, 3′-cyclic nucleotide 3′-phosphodiesterase) neuronal proteins useful for pre-Type 1 diabetes screening and/or staging.

### CROSS REFERENCE TO RELATED APPLICATION

The instant application is a continuation-in-part of application Ser. No. 09/954,972, filed on Sep. 17, 2001, the contents of which is herein incorporated by reference.

### FIELD OF THE INVENTION

This invention relates to autoimmune (Type 1A) diabetes mellitus (T1D). Specifically, the invention relates to the early diagnosis of pre-Type-1 diabetes based on the discovery that nervous system proteins play a role in early stage autoimmunity, particularly serving as markers of this process; and most particularly serving for the detection of nervous system proteins as the earliest harbingers of future disease risk and providing an unexpected, new target for intervention treatments.

### BACKGROUND OF THE INVENTION

Type 1 diabetes is an autoimmune disease of childhood that leads to a metabolic disorder characterized by abnormally high glucose levels in the bloodstream, and is associated with high subsequent risks of neurological, cardiovascular and other adverse health outcomes. In Type 1 diabetes the patient suffers from hyperglycemia due to a deficiency of insulin secretion. The incidence of Type 1 diabetes is highest for subjects between 9 and 13 years of age, with declining rates of incidence for subjects in their second and third decades (see Evidence-Based Diabetes Care, H. C. Gerstein and R. B. Haynes, editors, Hamilton, Ontario: BC Decker Inc., 2001). The prevalence of Type 1 diabetes is much higher in Caucasians than in Blacks, Asians or Hispanics, suggesting that genetic factors provide a significant contribution to the etiology of the disease (see Evidence-Based Diabetes Care, H. C. Gerstein and R. B. Haynes, editors, Hamilton, Ontario: BC Decker Inc., 2001). However, 85% of newly diagnosed patients do not have a family history of the disease. Environmental triggers have also been identified which are associated with the subsequent development of Type 1 diabetes, including dietary triggers, (e.g. discontinuation of breast-feeding before 3 months of age, high consumption of dietary nitrites in childhood) and viral infections (see Evidence-Based Diabetes Care, H. C. Gerstein and R. B. Haynes, editors, Hamilton, Ontario: BC Decker Inc., 2001).

Type 1 diabetes ranks as the fifth deadliest disease in the United States. The risk of developing Type 1 diabetes is higher than virtually all other chronic childhood diseases. Complications that affect every organ system can result from the pathology of diabetes and are classified either as microvascular complications (neuropathy, retinopathy and nephropathy) or macrovascular complications (cardiovascular disease). The total economic cost of diabetes in 2002 was estimated to be $132 billion in the United States (see Diabetes Care 26:917-932, American Diabetes Association, 2003).

Most of the currently available tests for Type I diabetes are blood tests that seek to confirm high blood sugar levels when compared with normal blood sugar levels. Urine-based tests are also used as screening tests for diabetes, since high blood sugar often results in sugar spilling into the urine. Currently used tests include: fasting plasma glucose, oral glucose tolerance, random plasma glucose and urine glucose (see the web site of the American Diabetes Association). The fasting plasma glucose test is the preferred way to diagnose diabetes, but the test is lengthy (12-14 hours) and has to be repeated at least once to confirm diagnosis. The oral glucose tolerance test requires fasting overnight and requires up to four collections of blood samples to confirm diagnosis. Results obtained from the random plasma glucose test are often inconclusive and unreliable. The urine glucose test is relatively easy to perform, but is considered less effective and precise than blood tests. Unfortunately, these described tests have little value for the detection of a pre-diabetic, clinically silent pre-symptomatic state(early pre-Type I diabetes). The ability to definitively diagnose this asymptomatic, early pre-Type 1 phase, is of tantamount importance, given that current immunotherapeutic modalities could effectively mitigate development of the disease.

There is an established genetic component in the etiology of Type 1 diabetes, as the risk of developing disease among siblings of affected individuals is approximately fifteen times that in the general population (Diabetologia 45:605-622 2002). The most important genes in terms of defining the risk for Type 1 diabetes, accounting for approximately one-half the genetic component, are located within the HLA gene complex (American Journal of Medical Genetics 115:30-36 2002). However, approximately 85% of new cases of Type 1 diabetes occur in individuals without a first degree relative with the disease, and even among monozygotic twins with a proband diagnosed with Type 1 diabetes at a very young age, only 38% go on to develop symptoms of the disease (Diabetologia 45:605-622 2002). Therefore, genetic testing alone appears to be inadequate as a general screening tool.

Screening of potential Type 1 diabetes subjects by immunoassay originally focused on islet cell autoantibodies and insulin autoantibodies, and was later expanded to include detection of autoantibodies for glutamic acid decarboxylase and tyrosine phosphatase. The role of these autoantibodies in assessing risk of conversion to Type 1 diabetes among healthy school children has been assessed in several prospective studies; in general, the risk of conversion to Type 1 diabetes is associated with the number of autoantibodies found to have elevated levels, however the predictive value of these immunoassays remains weak (Diabetes Care 25:505-511 2002; Diabetes 46:1701-1710 1997; Diabetologia 42:661-670 1999; Diabetes 45:926-933 1996; Journal of Clinical Endocrinology and Metabolism 87:4572-4579 2002 and Diabetes 48:460-468 1999). The appearance of insulin autoantibodies may occur earlier than glutamic acid decarboxylase or tyrosine phosphatase, but the ability of insulin autoantibodies to detect new-onset Type 1 diabetes cases may be weaker than that of any of the other autoantibodies (Diabetes 46:1701-1710 1997).

Currently, screening for genetic or immune markers of Type 1 diabetes is only recommended within the context of subject recruitment for well-defined research studies (Diabetes Care 24:398 2001). Indeed, the appearance of autoantibodies may occur during a stage of the clinical course of diabetes which is so far advanced that potential interventions are rendered ineffectual. The Diabetes Prevention Trial-Type 1 diabetes (DPT-1) was a randomized, controlled, non-blinded clinical study to determine the effect of insulin therapy in healthy first-degree relatives of Type 1 diabetes subjects who had been pre-screened and found to have high levels of islet cell autoantibodies. It was found that insulin therapy did not prevent or delay the onset of Type 1 diabetes relative to subjects who were merely kept under observation (New England Journal of Medicine 346:1685-1691 2002). The European Nicotinamide Diabetes Intervention Trial (ENDIT) was a randomized, placebo-controlled, double-blind clinical study performed on healthy first-degree relatives (FDR) of Type 1 diabetes subjects who had been pre-screened and found to have high levels of islet cell antibodies, the results of this trial were recently reported, and no significant difference was found between the nicotinamide and placebo groups in terms of 5-year cumulative risk of developing Type 1 diabetes (Diabetologia 46:339-346 2003). Thus, a need for an assay that can definitively diagnose pre-Type 1 diabetes still exists.

This need has re-kindled intense studies of prodromal autoimmunity in animal models. The NOD mouse (non-obese diabetic), as the premier animal model of Type 1 diabetes in humans, exhibits a polygenic autoimmune disease whose penetrance is under the control of environmental factors (M. Knip, H. K. Akerblom, Exp Clin Endocrinol Diabetes 107, S93-100 (1999); D. B. Schranz, A. Lernmark, Diabetes Metab Rev 14, 3-29 (1998); G. T. Nepom, W. W. Kwok, Diabetes 47, 1177-84 (1998); J. A. Todd, Pathol Biol (Paris) 45, 219-27 (1997); M. A. McAleer et al., Diabetes 44, 1186-1195 (1995)).

Insulin deficiency is the hallmark of diabetes and is the end result of a slowly progressive process, termed pre-diabetes, characterized by the accumulation of increasing amounts of dense T cell infiltrates first around (‘peri-insulitis’) and then eventually inside the islet (‘invasive insulitis’).

This slow progression and its biological controls are not well understood. Without ready access to the sparsely distributed islets in the human pancreas, most concepts of pre-diabetes progression derive from the rodent models of the disease (A. A. Rossini, E. S. Handler, J. P. Mordes, D. L. Greiner, Clin Immunol Immunopathol 74, 2-9 (1995); M. A. Atkinson, E. H. Leiter, Nat Med 5, 601-4 (1999)). However, there is strong consensus that human T1D is also characterized by the development of T-cells and autoantibodies that recognize β-cell constituents, the former are effectors of β-cell demise during a decade or more of clinically silent pre-diabetes.

Early NOD pre-diabetes has successfully been targeted by multiple immunotherapies that slow or altogether halt its progression to overt insulin deficiency and thus diabetes (M. A. Atkinson, E. H. Leiter, Nat Med 5, 601-4 (1999); S. Winer et al., J Immunol 165, 4086-4094 (2000); D. L. Kaufman et al., Nature 366, 69-72 (1993); R. Tisch et al., Nature 366, 72-75 (1993); J. Tian et al., Nature Med. 2, 1348-1353 (1996); J. Tian et al., J Exp Med 183, 1561-7 (1996); J. Tian, C. Chau, D. L. Kaufman, Diabetologia 41, 237-40 (1998); R. Tisch, R. S. Liblau, X. D. Yang, P. Liblau, H. O. McDevitt, Diabetes 47, 894-9 (1998); R. Tisch et al., J Immunol 166, 2122-2132 (2001); J. F. Elliott et al., Diabetes 43, 1494-1499 (1994)). These immunotherapies have all targeted specific autoimmune responses as measured by autoantibodies. The therapeutic effects of the particular autoantigens or relevant epitope peptide fragments from these molecules, derive from the route of application (usually systemically rather than locally), with mechanisms of pre-diabetes delay or cessation ascribed to clonal deletion, anergy induction and modifications of disease-associated cytokine bias. Unfortunately, the autoantibody responses targeted by these immunotherapies appear relatively late in pre-diabetes. R. B. Lipton et al., Amer J Epidemiol 136, 503-12 (1992); R. B. Lipton et al., Diabet Med 9, 224-32 (1992)). Treatments are effective only if applied earlier in pre-diabetes, while later treatments can precipitate overt disease (K. Bellmann, H. Kolb, S. Rastegar, P. Jee, F. W. Scott, Diabetologia 41, 844-847 (1998); R. Tisch, B. Wang, D. V. Serreze, J Immunol 163, 1178-1187 (1999); S. Winer et al., J Immunol 165, 4086-4094 (2000)).

Nevertheless, these observations have engendered optimism in the field that organ-selective autoimmune diseases such as T1D can be successfully prevented in humans at risk for the disease, by immunological interventions that modify the progression of early disease stages. In this, the pressing need for earlier diagnosis of diabetes risk is clear.

In the United States, these developments and needs have been acknowledged by considerable increases in funding for diabetes research, including the development of NIH-sponsored, $300 million research efforts such as THE IMMUNE TOLERANCE NETWORK, TRIGR and TRIALNET. These efforts are aimed at unifying strategies for the translation of animal data to human clinical intervention/prevention trials in organ-selective autoimmune diseases, with T1D the leading concern—reflecting its 100+ billion dollar annual cost in the US (˜80% of the total diabetes burden).

The past two decades of human T1D research had as its main theme the development of techniques that would allow reliable detection of prodromal disease states and pre-diabetes (W. Karges, et al., Molec Aspects Med 16, 79-213 (1995); D. B. Schranz, A. Lernmark, Diabetes Metab Rev 14, 3-29 (1998); R. B. Lipton et al., Amer J Epidemiol 136, 503-12 (1992); R. B. Lipton et al., Diabet Med 9, 224-32 (1992); C. F. Verge et al., Diabetes 45, 926-33 (1996); W. Woo et al., J Immunol Methods 244, 91-103. (2000)).

International workshops continue to provide important controls and improvements in these diagnostic efforts C. F. Verge et al., Diabetes 47, 1857-66 (1998); R. S. Schmidli, P. G. Colman, E. Bonifacio, and Participating Laboratories, Diabetes 44, 631-635 (1995); R. S. Schmidli, P. G. Colman, E. Bonifacio, G. F. Bottazzo, L. C. Harrison, Diabetes 43, 1005-9 (1994); N. K. MacLaren, K. Lafferty, Diabetes 42, 1099-1104 (1993)). However, while the accuracy of pre-diabetes diagnostics has improved, it is clear that present autoimmune serology detects only the mid- to late stages of the process with confidence. These stages are characterized in animal models as largely resistant to intervention, and immunotherapy at these stages can accelerate progression and precipitate overt disease (reviewed in S. Winer et al., J Immunol 165, 4086-4094 (2000)).

Thus, the need for very early detection of T1D-risk and impending diabetes is still pressing. While most current studies focus on families with the disease, such techniques must eventually be applicable to the general population, since 85% of new patients do not have a family history of autoimmune disease (W. Karges, J. Ilonen, B. H. Robinson, H.- M. Dosch, Molec Aspects Med 16, 79-213 (1995).

As discussed above, diagnostic tests presently available for Type 1 diabetes are cumbersome and expensive, and often identify the disorder so late in the disease that treatment options are limited. Early diagnosis of Type 1 diabetes should provide physicians, patients and families with enhanced treatment options. Both life style changes and the judicious use of currently available therapies may lead to improvement in patient outcomes when the diagnosis is made early. It is clear that if markers indicative of the earliest stages of pre-diabetes could be targeted, that a better understanding and staging of early pre-diabetes would be realized, and that therapeutic strategies and avenues capable of altering the course, progression and/or manifestation of the disease would be realized. The ability to identify markers of Type 1 diabetes at a very early stage in the clinical course of the disease is crucial in order to maximize the impact of intervention therapies aimed at preventing or stalling the onset of diabetes. Such markers of early pre-Type 1 diabetes are probably a prerequisite for successful human intervention trials.

### PRIOR ART

Poletaev et al. (Autoimmunity 32(1):33-38 2000) disclose that serum levels of natural autoantibodies of IgG class to proteins S100β, GFAP and NGF (nerve growth factor) are higher in patients suffering from various neurological disorders (depressive disorder, epilepsy, multiple sclerosis, Parkinson's disease) than in healthy adult patients. Poletaev et al. interpret from these results that changes in the mechanisms of the immune state represent the common features of different forms of pathology of the nervous system.

Górny et al. (Neurologia I neurochirurgia polska 24:17-20 1990) disclose that levels of autoantibodies for GFAP in the cerebrospinal fluid of patients suffering from multiple sclerosis and neurological disorders were higher as compared with a control group.

Ishida et al. (Journal of Neurological Sciences 151:41-48 1997) disclose the finding of an autoantibody for GFAP in the serum of a patient suffering from dementia and an autoimmune disorder.

Hagopian et al. (U.S. Pat. No. 5,547,847) disclose a diagnostic assay which tests for the presence of autoantibodies for human islet cell glutamic acid decarboxylase (GAD64) in blood products (blood, plasma and serum). Based upon the presence or absence of autoantibodies for GAD, patients can be classified as to the predicted course of the disease. The assay of Hagopian et al. is particularly useful to distinguish between insulin-dependent diabetes (Type 1) and non-insulin dependent diabetes (Type 2) since many patients who are initially diagnosed as having Type 2 diabetes actually have Type 1.

Rabin et al. (U.S. Pat. No. 5,200,318) disclose an assay for diagnosing Type 1 diabetes using a panel of immunoreagents. The immunoreagents comprise two or more epitopes of GAD and islet cell antigens ICA512 and ICA12. The assay of Rabin et al. is used to capture antibodies for the immunoreagents from a patient blood sample in order to screen for pre-Type 1 diabetes, distinguish Type 1 diabetes from Type 2 diabetes and to monitor therapy.

Tobin et al. (U.S. Pat. No. 6,455,267 B1) disclose GAD polypeptides which are useful for diagnosing and ameliorating GAD-associated autoimmune diseases. The method of Tobin et al. relies on contacting T cells from a patient with a GAD polypeptide and detecting the response of the T cells wherein a T cell response indicates the presence of a GAD-associated autoimmune disorder. Tobin et al. also disclose that their GAD polypeptides can be used in immunoassays to detect antibodies to the GAD polypeptides.

While these studies focus on either nervous system protein markers or their corresponding autoantibodies, the instant inventors focus on both nervous system protein markers (antigens) and their corresponding autoantibodies, particularly those associated with the islet cells. It is particularly important to focus on the nervous system protein markers because in theory these markers should appear in the serum prior to the appearance of the autoantibodies and thus, offer an even earlier indication of impending disease. The instant inventors are the first to recognize that damage to the neuronal tissue surrounding the islets occurs before the onset of diabetes and that neuronal protein markers and their corresponding autoantibodies indicative of this damage can be utilized for identification of individuals at high risk to develop Type-1 diabetes before symptoms occur.

### SUMMARY OF THE INVENTION

Type I diabetes is generally classified as a pediatric/adolescent disease wherein an autoimmune response results in progressive β cell destruction, insulin deficiency and hyperglycemia. It has long been felt that the manifestation of the disease is preceded by a clinically silent phase known as “pre-Type 1 diabetes”. However, there has, heretofore, been no practical method for accurately diagnosing this early phase of the disease process.

The ability to definitively diagnose this asymptomatic, pre-type I diabetes phase is of tantamount importance, given that current immunotherapeutic modalities could effectively mitigate development of the disease.

The instant inventors were the first to realize that the islet cell-associated neuronal tissue (Schwann cells which encapsulate the islets) was destroyed by auto-immune responses prior to the onset of insulitis (Winer et al. Nature Medicine 9(2):198-205; U.S. application Ser. No. 09/954,972; filed Sep. 17, 2001). In the earlier work of the instant inventors in diagnosing autoimmune conditions such as Type 1 diabetes, the instant inventors recognized that a loss of self-tolerance of a Schwann cell protein could be evidenced by a diagnostic marker comprising a binding protein indicative of such loss, particularly an autoantibody or immunologically detectable fragment thereof capable of recognizing an epitope of Schwann cell breakdown. Exemplary of such a diagnostic marker is an autoantibody which recognizes an epitope of GFAP.

Diagnostic testing utilizing autoantibodies alone can be problematic, given that there is often a background reading of autoantibodies in the general population.

Thus, the instant inventors theorized that as a precursor to the development of autoantibodies, the actual antigen, e.g. nervous system or neuronal tissue and degradation products thereof, must have previously appeared in the circulation when released from the damaged Schwann cells.

Since neuronal tissue, found to encapsulate the β cells, is destroyed during the pathogenic process of pre-Type I diabetes, it was theorized that circulating antigens concomitant with neuronal tissue destruction, could provide a readout of ongoing, slowly progressive pre-Type I diabetes.

The instant inventors have found it possible to quantify the direct pathology of pre-Type I diabetes tissue destruction, by looking at certain neuronal markers, particularly NSE and S100β.

These markers, particularly NSE and S100β, are not commonly found in the circulation of the pediatric/adolescent age group which is the intended target group of this diagnostic procedure.

Generally, the method of the instant invention provides a diagnostic test based on detection of at least one neuronal tissue marker and/or at least one autoantibody for a neuronal tissue marker and/or combinations of neuronal tissue markers and autoantibodies for neuronal tissue markers which will allow sensitive and specific prediction of an individual's propensity to develop Type I diabetes. These markers and autoantibodies are identified from body fluid samples and can be analyzed by using any of the known immunoassays. Illustrative, albeit non-limiting, examples of immunoassays are sandwich, radioimmunoassay, fluorescent or chemiluminescence immunoassay, and immunoPCR technology. A particularly preferred immunoassay is the sandwich immunoassay.

Accordingly, it is an objective of the instant invention to identify neuronal protein marker antigens or fragments thereof indicative of a loss of self-tolerance to the nervous system tissue of the pancreas.

It is another objective of the instant invention to identify autoantibodies for neuronal protein marker antigens or fragments thereof indicative of a loss of self-tolerance to the nervous system tissue of the pancreas.

It is a further objective of the instant invention to provide a method for diagnosing and staging pre-Type I diabetes by analyzing a body fluid sample for the clinically relevant presence of at least one neuronal tissue marker or fragments thereof including; GFAP (glial fibrillary acidic protein); NSE (neuron specific enolase); GAD65 (glutamic acid decarboxylase); S100β and CNPase (2′, 3′-cyclic nucleotide 3′-phosphodiesterase).

It is yet a further objective of the instant invention to provide a method for diagnosing and staging pre-Type I diabetes by analyzing a body fluid sample for the clinically relevant presence of at least one autoantibody for a neuronal tissue marker or fragments thereof including autoantibodies for; GFAP (glial fibrillary acidic protein); NSE (neuron specific enolase); GAD65 (glutamic acid decarboxylase); S100β and CNPase (2′, 3′-cyclic nucleotide 3′-phosphodiesterase).

It is yet a further objective of the instant invention to provide a method for diagnosing and staging pre-Type I diabetes by analyzing a body fluid sample for the clinically relevant presence of combinations of neuronal tissue markers or fragments thereof; combinations of autoantibodies for neuronal tissue markers or fragments thereof and combinations of both neuronal tissue markers and fragments thereof and autoantibodies for neuronal tissue markers and fragments thereof.

It is yet another objective of the instant invention to provide a diagnostic assay test kit based on detection of at least neuronal tissue marker or fragments thereof which will allow sensitive and specific prediction of an individual's propensity to develop Type I diabetes.

It is another objective of the instant invention to provide a diagnostic assay test kit based on detection of at least one autoantibody for a neuronal tissue marker or fragments thereof which will allow sensitive and specific prediction of an individual's propensity to develop Type I diabetes.

It is still another objective of the instant invention to provide a diagnostic assay test kit based on detection of combinations of neuronal tissue markers or fragments thereof, combinations of autoantibodies for neuronal tissue markers or fragments thereof and/or combinations of neuronal tissue markers or fragments thereof and autoantibodies for neuronal tissue markers or fragments thereof which will allow sensitive and specific prediction of an individual's propensity to develop Type I diabetes.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

The instant patent or application file contains at least one drawing executed in color. Copies of this patent or patent application publication with color drawing(s) will be provided by the Office upon request and payment of the necessary fee.

FIG. 1 illustrates a SELDI process using GFAP-coupled chip arrays;

FIG. 2 illustrates the presence of GFAP binding protein in 4 week old NOD female mice;

FIG. 3 illustrates a comparison of male vs. female NOD mice at 5 weeks;

FIGS. 4A-D illustrate a comparison of serum samples from patients with recent onset T1D (FIG. 4B), from autoantibody-positive first degree relatives with probable pre-diabetes (FIG. 4A) and from relatives without signs of autoimmunity (FIGS. 4C, D), which were analyzed in a similar fashion as NOD mice.

FIG. 5 is an electron micrograph of pancreatic tissue showing exocrine tissue and a Schwann cell surrounding an islet.

FIG. 6 is a micrograph obtained with a scanning electron microscope of a peri-islet Schwann cell with surrounding neurons.

FIG. 7 shows that the pancreatic islets of Langerhans are surrounded by Schwann cells through the use of immunohistochemistry and microscopy.

FIG. 8 shows the destruction of peri-islet Schwann cells early in pre-diabetes through the use of immunohistochemistry and microscopy.

FIG. 9 shows that Schwann cells are completely destroyed in diabetic islets through the use of immunohistochemistry and microscopy.

FIG. 10 is a graph showing that T-cell autoreactivity to Schwann cell antigens occurs early in non-obese diabetic (NOD) mice.

FIGS. 11A-C show the detection of antibody against Schwann cell antigens in sera from NOD mice. Figure A shows results obtained through the use of RT-PCR. Figure B shows results obtained through the use of a western blot. Figure C shows results obtained through the use of mass spectrometry.

FIG. 12 shows the detection of antibody against Schwann cell antigens in sera from diabetic humans through the use of mass spectrometry.

FIG. 13 is a graph showing the prediction of risk for the development of diabetes based on Schwann cell autoimmunity.

FIG. 14 shows that antibodies obtained from pre-diabetic children react with peri-islet Schwann cells through the use of immunohistochemistry and microscopy.

FIG. 15 is a schematic illustrating adoptive diabetes transfer in a mouse model system.

FIG. 16 is a schematic illustrating that immunotherapy prevents the development of diabetes in the adoptive diabetes transfer mouse model system.

FIG. 17 is a graph showing results of immunotherapy using purified protein.

FIG. 18 is a graph showing a GFAP epitope map.

FIG. 19 is a graph measuring Schwann cell autoimmunity in human diabetes.

FIGS. 20A-B are graphs showing results obtained from ELISA assays; FIG. 20A shows S100β levels in blood samples by subject classification and FIG. 20B shows NSE levels in blood samples by subject classification (clinical samples (FDR) obtained from ENDIT study).

FIG. 21 shows a diagram illustrating the conventional view of the natural history of diabetes.

FIG. 22 shows a diagram illustrating the natural history of diabetes as revised by the instant inventors.

FIG. 23 shows a diagram illustrating the anticipated biomarker levels along the course of Type-1 diabetes as seen in the context of the natural history of diabetes as revised by the instant inventors.

### Definitions and Abbreviations

The following list defines terms, phrases and abbreviations used throughout the instant specification. Although the terms, phrases and abbreviations are listed in the singular tense the definitions are intended to encompass all grammatical forms.

As used herein, the term “early pre-Type 1 diabetes” refers to the asymptomatic phase of Type-1 diabetes occurring prior to the clinical onset of Type-1 diabetes.

As used herein, the phrase “clinically relevant” refers to an amount (for example, of a neuronal tissue marker or of an autoantibody for a neuronal tissue marker) which is sufficient to distinguish an individual at high risk for the development of Type-1 diabetes from age-matched normal individuals within the target population.

As used herein, the phrase “at-risk population” refers to the pediatric/adolescent population that Type I diabetes commonly affects.

As used herein, the phrase “target population” refers to those individuals of the at-risk population and their first degree relatives (regardless of age) having elevated levels of ICA autoantibodies. First degree relatives (FDR) usually range from 3-40 years in age.

As used herein, the term “fragment” refers to a polypeptide sequence truncated or shortened in length as compared with the length of the polypeptide designating the complete protein; such “fragments” are immunologically detectable.

As used herein, the term “corresponding autoantibody” or “corresponding antibody” refers to a binding protein which recognizes an epitope(s) of a specific antigen; for example, a protein which binds an antigen is the corresponding antibody of that particular antigen.

As used herein, the term “combinations” refers to groups of two or more neuronal tissue markers selected from the group consisting of GFAP, GAD65, NSE, S100β and CNPase; or groups of two or more autoantibodies for neuronal tissue markers selected from the group consisting of autoantibodies for GFAP, GAD65, NSE, S100β and CNPase; or groups of at least one neuronal tissue marker selected from the group consisting of GFAP, GAD65, NSE, S100β and CNPase and at least one autoantibody for a neuronal tissue marker selected from the group consisting of autoantibodies for GFAP, GAD65, NSE, S100β and CNPase.

As used herein, the abbreviation “T1D” refers to Type 1 diabetes or insulin-dependent diabetes mellitus.

As used herein, the abbreviation “MS” refers to multiple sclerosis.

As used herein, the abbreviation “SC” refers to Schwann cells.

As used herein, the abbreviation “GFAP” refers to glial fibrillary acidic protein.

As used herein, the abbreviation “NSE” refers to neuron specific enolase.

As used herein, the abbreviation “GAD65” refers to glutamic acid decarboxylase.

As used herein, the abbreviation “CNPase” refers to 2′, 3′-cyclic nucleotide 3′-phosphodiesterase.

As used herein, the abbreviation “NGF” refers to nerve growth factor.

As used herein, the abbreviation “ICA” refers to islet cell antibodies.

As used herein, the abbreviation “HLA” refers to human leukocyte antigen.

As used herein, the abbreviation “NOD” refers to non-obese diabetic mouse which is the premier animal model of human Type-1 diabetes.

As used herein, the abbreviation “FDR” refers to first degree relative (of a patient having T1D); an individual who may be at greater risk for developing T1D.

As used herein, the abbreviation “PBS” refers to phosphate buffered saline.

As used herein, the abbreviation “SELDI-TOF MS” refers to surfaces enhanced for laser desorption/ionization time-of-flight mass spectrometry.

As used herein, the abbreviation “ELISA” refers to an enzyme-linked immunosorbent assay. An ELISA assay is also referred to as a “sandwich” assay.

The terms “neuronal tissue marker” and “nervous system tissue marker” are used interchangeably herein.

The term “neuronal proteins” as used herein, refers to a neuronal antigen, a neuronal autoantibody or both a neuronal antigen and a neuronal autoantibody.

The terms “T1D”, “diabetes mellitus” and “insulin-dependent diabetes” are used interchangeably herein to refer to Type-1 diabetes.

### DETAILED DESCRIPTION OF THE INVENTION

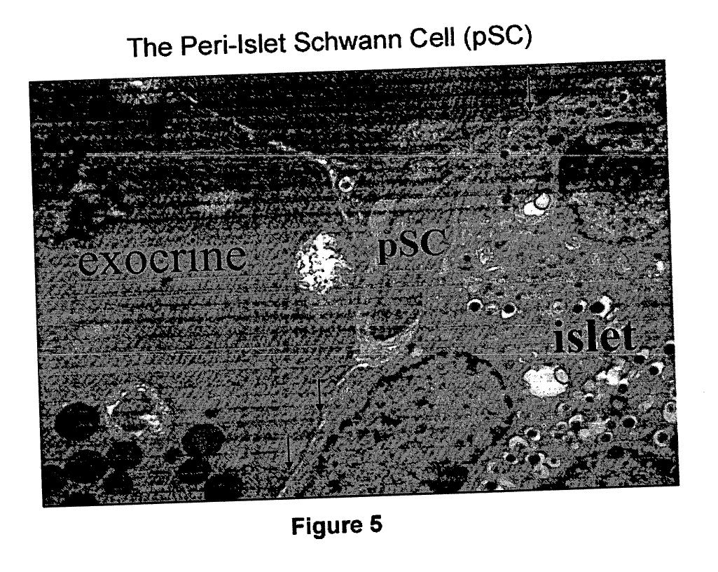

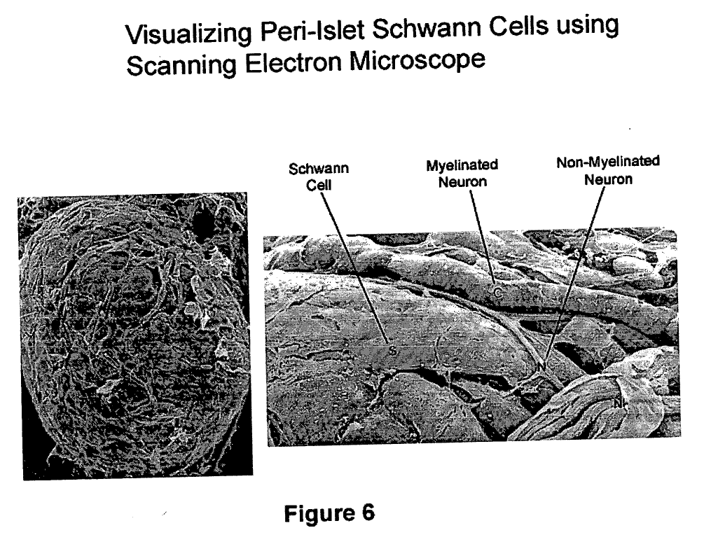

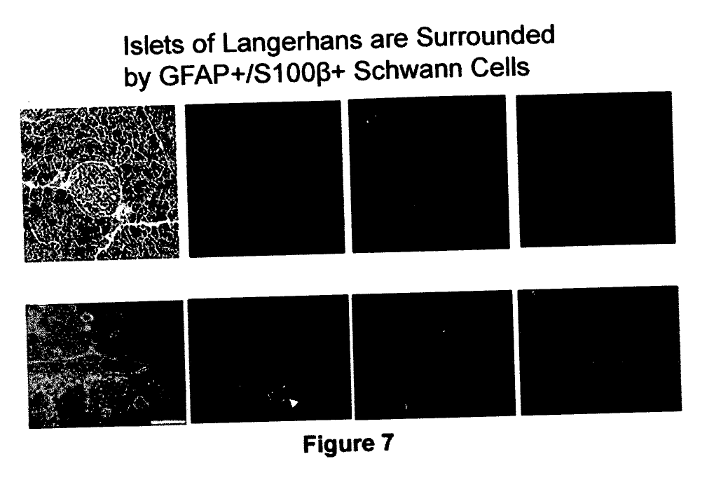

The mature pancreas exhibits both exocrine (secretion of enzymes) and endocrine (secretion of hormones) functions. The exocrine pancreatic tissue produces digestive enzymes and the islets of Langerhans (endocrine pancreas) produce hormones, such as insulin, glucagon and somatostatin. Insulin and glucagon regulate blood glucose levels. Schwann cells, the pancreatic neuronal tissue, surround each islet. FIG. 5 represents an electron micrograph of a pancreatic tissue section showing exocrine tissue, an islet and a Schwann cell surrounding the islet (peri-islet Schwann cell). FIG. 6 represents a micrograph of a peri-islet Schwann cell as imaged by a scanning electron microscope. This micrograph shows the cellular surface crisscrossed by both myelinated and non-myelinated neurons. FIG. 7 represents a series of micrographs stained using immunohistochemical techniques which illustrate that the pancreatic islets are surrounded by Schwann cells. These Schwann cells are GFAP⁺ (identified by the green stain) and S100β⁺ (data not shown). The blue stain identifies insulin.

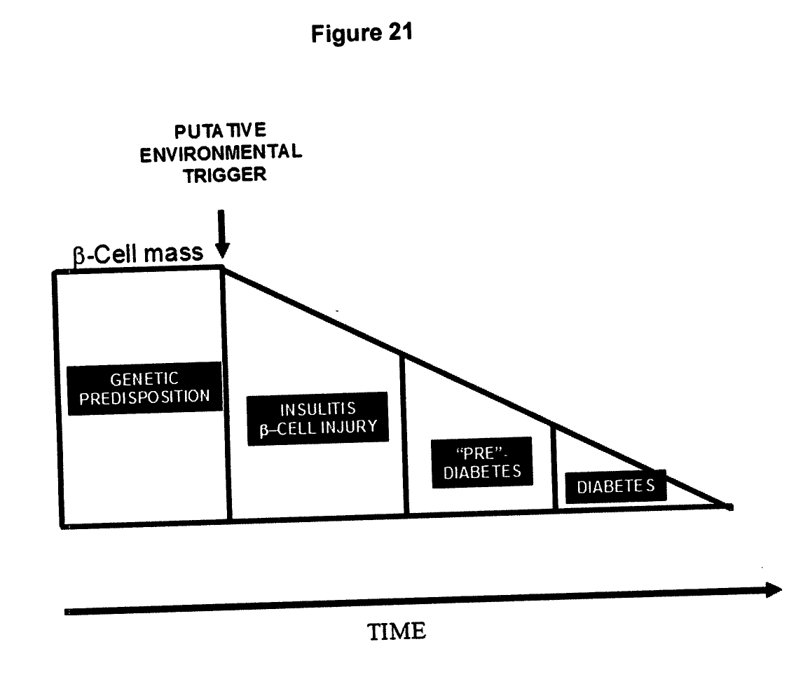

Type 1 diabetes is an autoimmune response which results in destruction of the pancreatic β cells leading to insulin deficiency and hyperglycemia. FIG. 21 shows a diagram illustrating the conventional view of the natural progression of diabetes. The number of functioning pancreatic β cells are progressively reduced over time ultimately resulting in the clinical onset of diabetes (diagram adapted from Schatz et al. Hormone Research 57(1):12-17 2002). β cell destruction is initiated by an environmental trigger in an individual having a genetic predisposition for developing diabetes. β cell injury occurs due to the production of humoral autoantibodies (for example, but not limited to, ICA, IAA, GAD65 and ICA512) for pancreatic cell components. During this period, additional injury occurs due to the onset of cellular T-cell autoimmunity (also for pancreatic cell components). The auto-reactive T-cells first surround the islets and eventually invade the interior of the islets resulting in the progressive loss of functioning β cells. A progressive decrease in insulin production occurs simultaneously with, and as a result of β cell destruction. At this stage, diagnosis of the diabetic condition is frequently attempted using conventional tests such as, IVGTT (intravenous glucose tolerance test) and OGTT (oral glucose tolerance test). Patients experiencing a loss of the first phase insulin response are often labeled “pre-diabetic”; however symptoms heralding the clinical onset of diabetes frequently occur shortly after this diagnosis. Treatment is often not effective in the late stages of the disease (pre-diabetic and symptomatic diabetes) since the islets are completely or near completely destroyed. Diagnosis in the earlier stages would improve treatment options and therapeutic results for many patients.

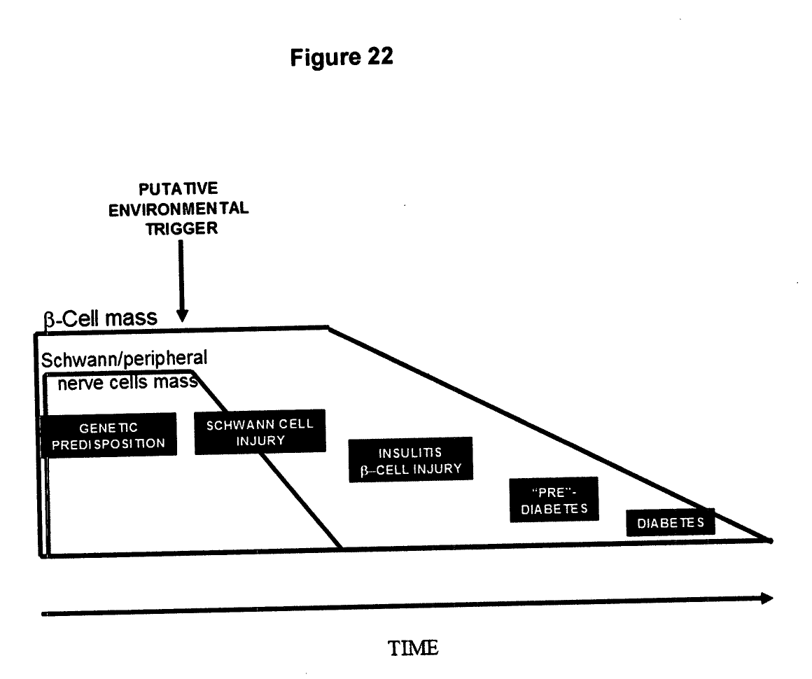

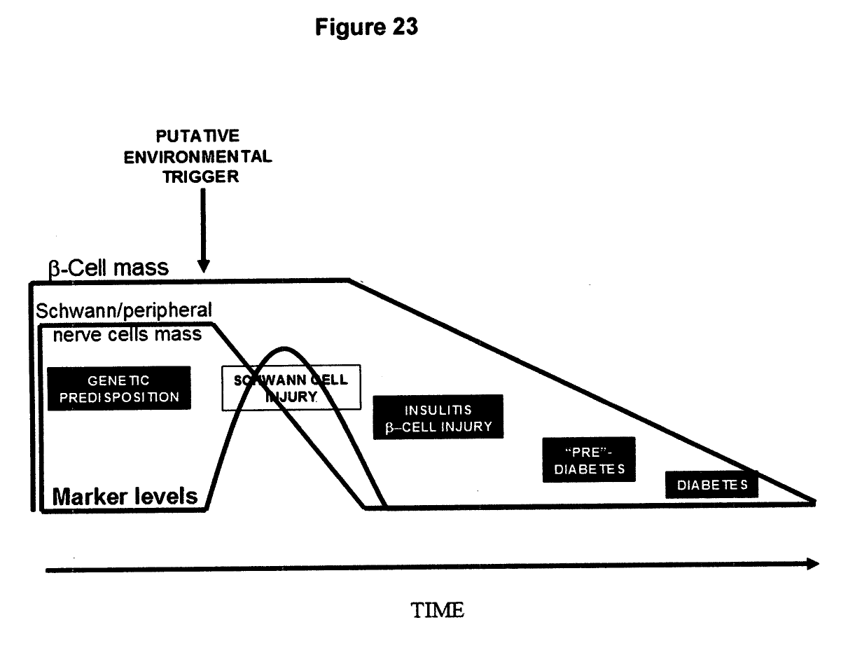

The instant invention relates to the early diagnosis of pre-Type-1 diabetes based on the discovery that neuronal proteins play a role in early stage auto-immunity. FIG. 22 shows a diagram illustrating the natural progression of diabetes as theorized by the instant inventors. The instant inventors recognized that a phase of nerve cell injury precedes the onset of insulitis. The damage occurs to the Schwann cells; the nervous tissue mass surrounding the islets. During this phase of Schwann cell injury there is a release of neuronal tissue biomarkers into the circulation. The instant inventors were the first to recognize that if diagnostic assays could be developed based on the detection of these neuronal tissue biomarkers; it may be possible to improve treatment options and/or delay the clinical onset of diabetic symptoms based on early disease detection prior to β cell destruction. FIG. 23 shows a diagram illustrating the natural progression of diabetes as theorized by the instant inventors including the anticipated levels of biomarkers released into the circulation during the phase of Schwann cell injury. Subsequent to the release of such biomarkers, autoantibodies for the biomarkers are produced.

The location of expression of the biomarkers was investigated to determine tissue of origin. The biomarkers were found to be of neuronal origin. Neuron specific enolase (NSE) is found only in neurons. Glutamic acid decarboxylase (GAD65) is found in both peripheral and central nervous system tissue. S100β is found in neurons, Schwann cells and in trace amounts in β-cells. GFAP is found only in Schwann cells. CNPase is found in the myelin of the central nervous system.

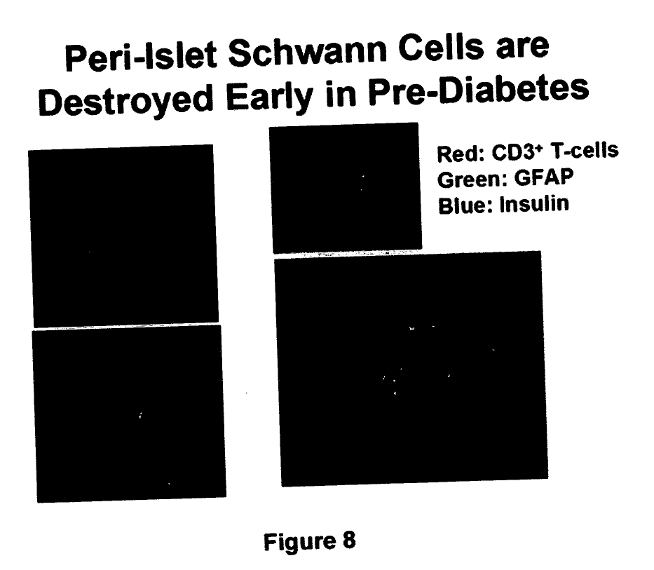

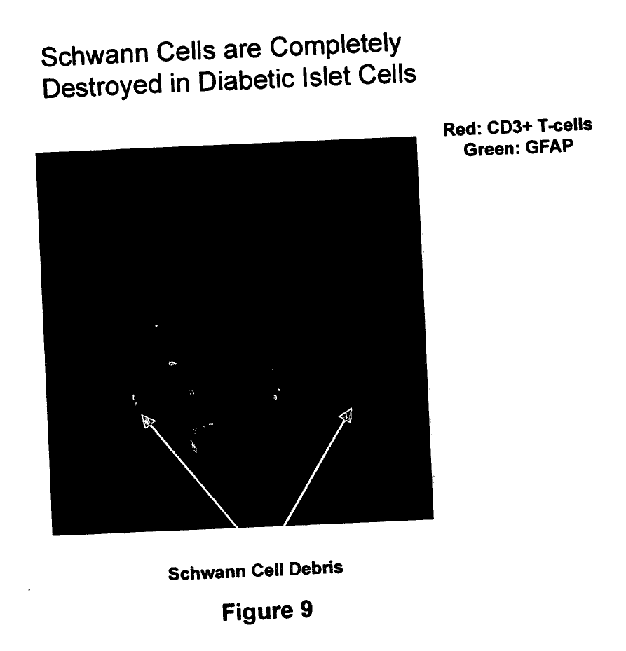

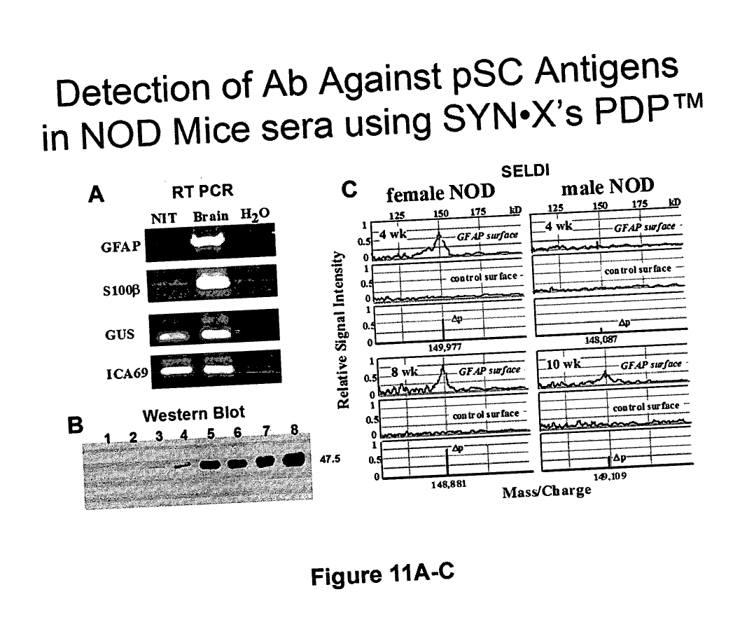

Other pancreatic cells, in addition to the Schwann cells, were tested to determine if expression of the biomarkers was limited to neuronal tissue. FIG. 11A shows the results of RT-PCR used to determine gene expression in NITβ cells (an islet cell line). GFAP expression is exclusive to Schwann cells in the pancreas and is not expressed by cells of any other pancreatic tissue. Thus, GFAP is a particularly useful marker for indication of the Schwann cell destruction which occurs early in pre-diabetes. FIG. 8 shows a series of micrographs stained using immunohistochemical techniques which evidence the destruction of peri-islet Schwann cells early in pre-diabetes. The red stain identifies CD3⁺ T-cells (auto-reactive), the green stain identifies GFAP and the blue stain identifies insulin. FIG. 9 shows a micrograph stained using immunohistochemical techniques which evidences the complete destruction of the peri-islet Schwann cells in diabetes. The red stain identifies CD3⁺ T-cells and the green stain identifies GFAP. Additionally, since β-cells (cells of the islet) themselves express trace amounts of GAD65 as well as S100β, but lack GFAP expression detectable by RT-PCR (see FIG. 11A), GFAP provides a local Schwann cell marker. FIG. 11A shows GFAP transcripts are detectable by template-calibrated RT-PCR in brain but not in the NIT β-cell line, which expresses trace amounts of S100β and large amounts of ICA69 (islet cell cytoplasmic auto-antibodies). β-glucuronidase was used for calibration.

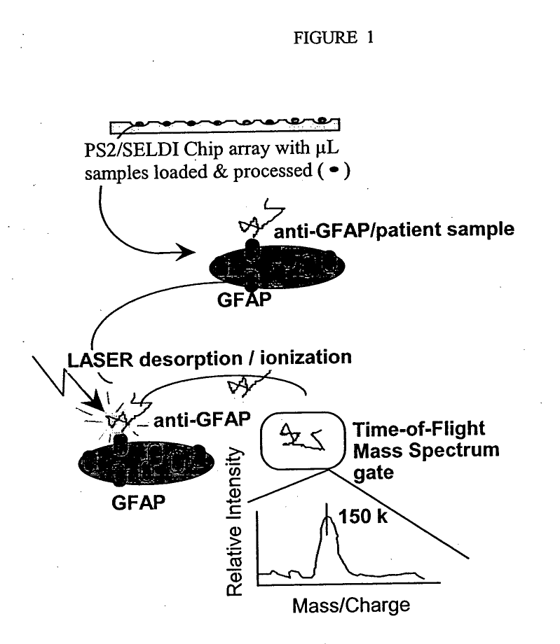

With reference to FIG. 1, IgG autoantibodies for GFAP were measured in sera from NOD mice of different ages, using covalently GFAP-coupled chip arrays in a SELDI-time-of-flight mass spectrometry instrument calibrated with a monoclonal anti-GFAP antibody.

FIG. 11B demonstrates results obtained from a western blot used to detect antibodies for GFAP in mouse sera. An amount of 0.5 μg of GFAP was loaded in each lane. Lane 1 contains a control sample from 8 week old C57BL/6 mice, lanes 2-7 contain sera (1:150) from NOD female mice at ages 3.5 (lane 2), 5 (lane 3), 6 (lane 4), 8 (lane 5), 10 (lane 6) and 20 weeks old respectively. Lane 8 contains 1:1000 IgG GFAP antibody. This western blot evidences that female NOD mice have a GFAP auto-antibody detectable at 5 weeks of age.

FIG. 11C shows the results of a mass spectrometric experiment used to detect antibodies for GFAP in male and female NOD mouse sera. Covalently GFAP-coupled proteomic chip arrays were incubated with sera from 3 to 10-week-old NOD females or males as indicated. Chips were washed and read in a SELDI time of flight mass spectrograph. Control chip surfaces were identical except for the absence of GFAP. The differential peak signal (Δp) is shown in the bottom of the figures. Large peaks at 150 kD (IgG) were observed only in female sera. Four of 31 similar profiles are shown in this figure. This experiment shows that female NOD mice have a GFAP auto-antibody detectable at 4 weeks of age whereas male NOD mice do not have detectable GFAP auto-antibody even at 10 weeks of age. These results are consistent with the fact that female NOD mice develop diabetes much more frequently than male NOD mice.

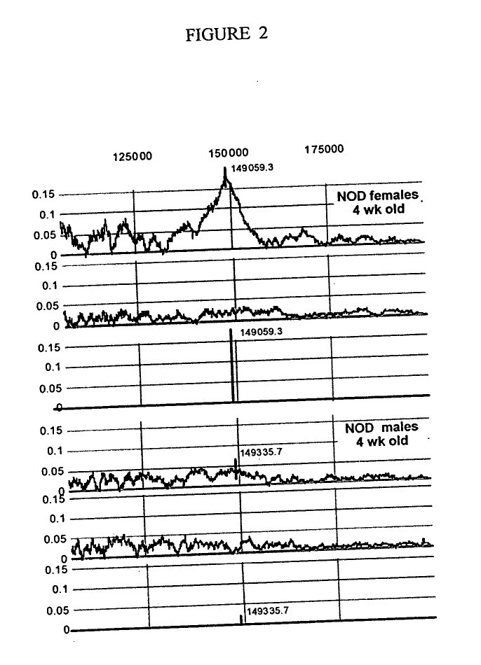

As seen in FIG. 2, serum from 11/13 NOD females as young as 4 weeks old contained a GFAP-binding protein of 149,805.71200 D mass. This 150 kD protein was removed by prior serum passage over solid phase GFAP or solid phase Protein G columns and thus represents IgG autoantibody. These autoantibodies were maintained in overtly diabetic mice 20-26 weeks of age. Samples with high autoantibody signals in SELDI-TOF-MS were found to contain anti-GFAP autoantibodies in Western blots, but the sensitivity of SELDI exceeds that of Western blots.

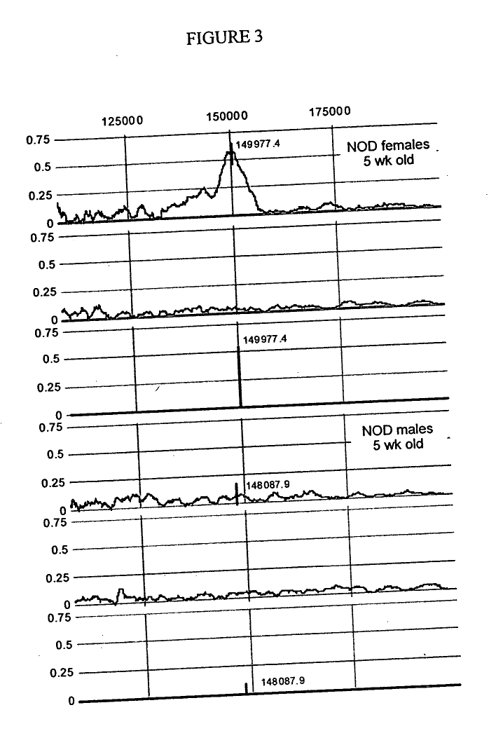

As set forth in FIG. 3, sera from male NOD mice 5-18 weeks of age, from 7 week old non-autoimmune strain C57Bl/6 and 8 week old Balb/c mice, or from NOD females 3 weeks of age were negative, while 5/8 samples from 4-5 week old females were clearly positive for GFAP autoantibodies.

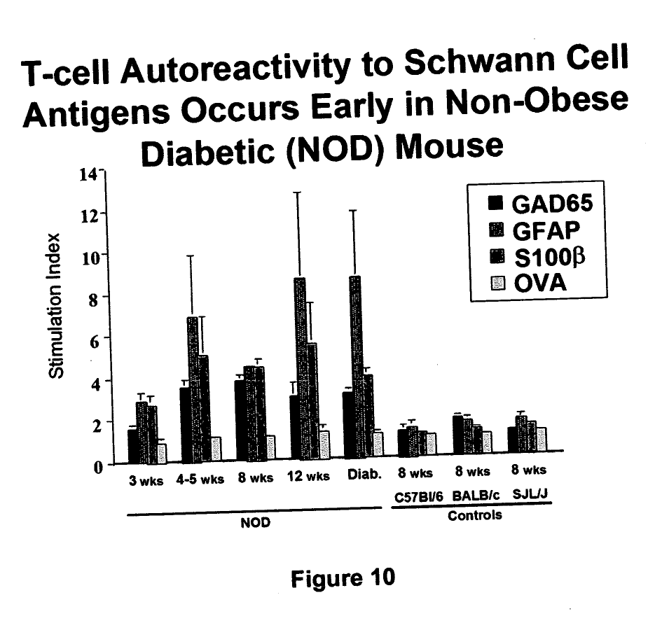

FIG. 10 is a graph showing results of an assay measuring T-cell autoreactivity in NOD mice. NOD mice were tested at 3, 4-5, 8 and 12 weeks of age respectively. NOD mice with overt diabetes were also tested. The control strains of mice were 8 week old C57BL/6, BALB/c and SJL/J mice. Autoreactivity of T-cells was tested against GAD65, GFAP and S100β protein antigens. OVA protein antigen was used as a control. The results evidence that GFAP and S100β are statistically significant indicators of pre-diabetes.

It was therefore concluded that loss of self-tolerance to the Schwann cell protein, GFAP, and likely other SC constituents such as S100β, is a characteristic of NOD mouse pre-diabetes and predicts the progressive disease course leading to overt T1D in female mice. There is no presently available serum marker to predict disease risk or overt disease in NOD mice before establishment of invasive insulitis by 10-12 weeks of age (S. Reddy, N. Bibby, R. B. Elliott, Clin Exp Immunol 81, 400-5 (1990)): in the case of NOD females GFAP autoantibodies have a positive predictive power of about 90% at an age of 5 weeks, i.e. before insulitis is established. This is an age where intervention therapies have the best effectiveness (discussed in: (S. Winer et al., J Immunol 165, 4086-4094 (2000); M. A. Atkinson, E. H. Leiter, Nat Med 5, 601-4 (1999)).

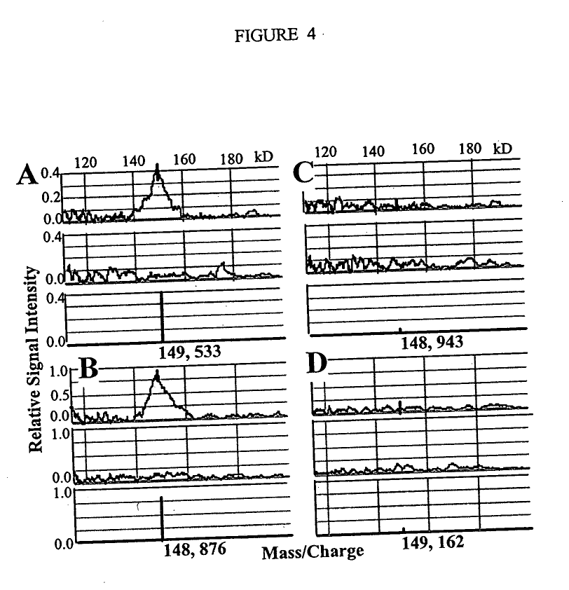

Diabetes-associated autoimmunity in NOD mice and humans targets a closely similar set of autoantigens. As seen in FIG. 4 (4A, 4B, 4C and 4D) serum samples from patients with recent onset T1D (FIG. 4B), from autoantibody-positive first degree relatives with probable pre-diabetes (FIG. 4A) and from relatives without signs of autoimmunity (FIGS. 4 C, D) were analyzed in a similar fashion as NOD mice. Samples from 24/30 new onset patients, 9/10 relatives with probable pre-diabetes 2/29 healthy controls, and 4/5 patients with probable MS contained anti-GFAP autoantibodies detected by SELDI-TOF-MS.

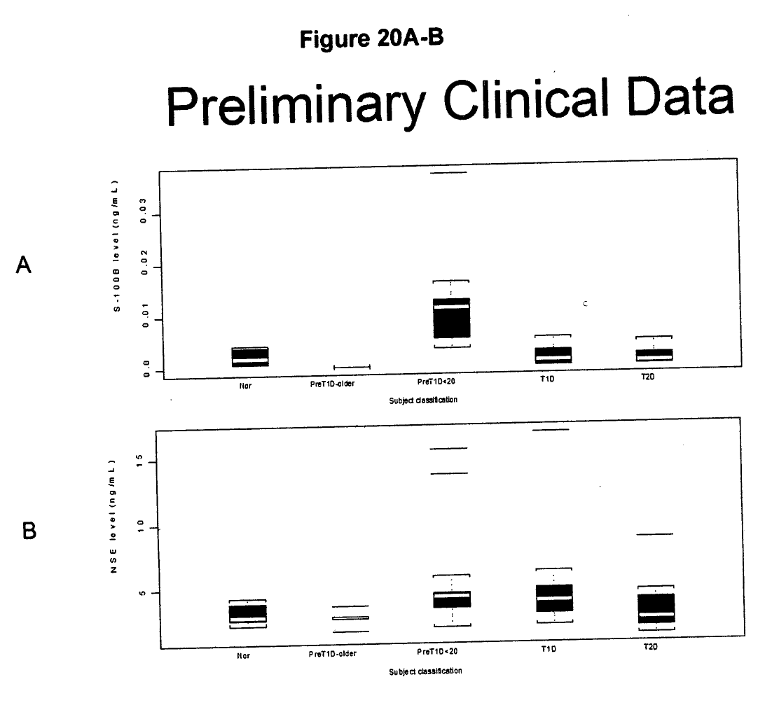

FIGS. 20A and 20B show data derived by use of samples from the European Nicotinamide Diabetes Intervention Trial; ENDIT (Diabetologia 46:1033-1038 2003). ENDIT was a randomized, double-blind, placebo-controlled intervention trial which was developed in order to determine whether nicotinamide therapy could prevent or delay onset of Type I diabetes in subjects between 3-40 years of age who had a first-degree family history of Type I diabetes and elevated ICA levels (greater than or equal to 20JDF units). ENDIT clinical samples from the study site in London, Ontario, Canada were obtained through a collaboration with Dr. HM Dosch; of these samples, 13 were pre-diabetics under the age of 20 who later converted to Type I diabetes and 5 were pre-diabetics 20 years or older who later converted to Type I diabetes. The samples obtained from patients involved in the ENDIT study were clinically relevant for testing the method of the instant invention, since it is known that patients from which samples were obtained did later convert to Type 1 diabetes, an evaluation of markers in their serum prior to conversion provides valuable insight to conditions of pre-Type 1 diabetes. In addition, samples were obtained from other screening studies at the same site, including 12 confirmed Type I diabetics, 8 confirmed Type II diabetics and 6 healthy non-diabetic adults. Serum S100β and NSE levels for all subjects were determined using the S100β and NSE ELISA test kits. FIG. 20A shows the S100β levels by subject classification. S100β appears to a marker specific to the pre-diabetic state in subjects under 20 years old; S100β levels are significantly higher in these subjects than in the normal subjects (p=0.002, Wilcoxon rank sum test). FIG. 20B shows NSE levels by subject classification. Preliminary results are also suggestive of NSE levels being higher in the young pre-diabetic subjects than in the normal subjects (p=0.072, Wilcoxon rank sum test). The biomarker levels (NSE and S100β) are elevated in patients less than 20 years of age, 2-4 years prior to onset of Type I diabetes. Thus, serum levels of these markers appear to be an effective diagnostic tool for identification of individuals at high risk for development of Type I Diabetes such that treatment can be administered before the onset of symptoms.

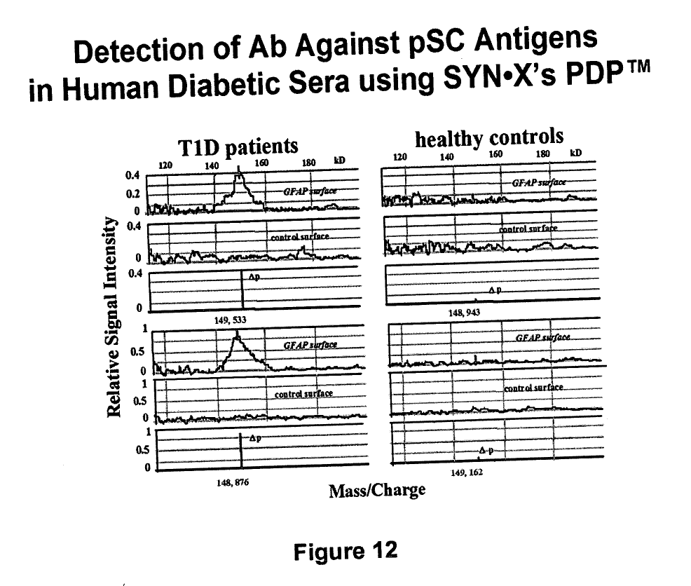

FIG. 12 shows the results of a mass spectrometric experiment (SELDI) used to detect autoantibody for Schwann cell antigens in human diabetic sera. The GFAP auto-antibody was found in T1D patients and absent from healthy patients (control). The differential peak signal (Δp) is shown on the bottom of the figures.

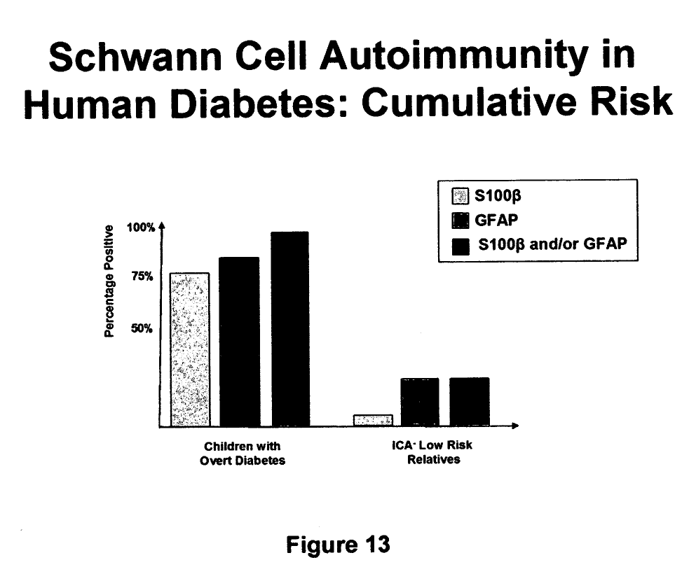

FIG. 13 shows a chart predicting the cumulative risk for the development of diabetes in the relatives of children with overt diabetes. The prediction was made based on a test of T-cell response to S100β and GFAP proteins. The chart compares the percentage of T-cell responses in children having overt diabetes to the percentage of T-cell responses in low risk relatives of the children. The group of low risk relatives test negative for the presence of ICA (islet cell antibodies). The yellow bar measures the T-cell responses to the S100β protein; the green bar measures the T-cell responses to the GFAP protein and the red bar measures the T-cell responses to S100β and/or GFAP proteins.

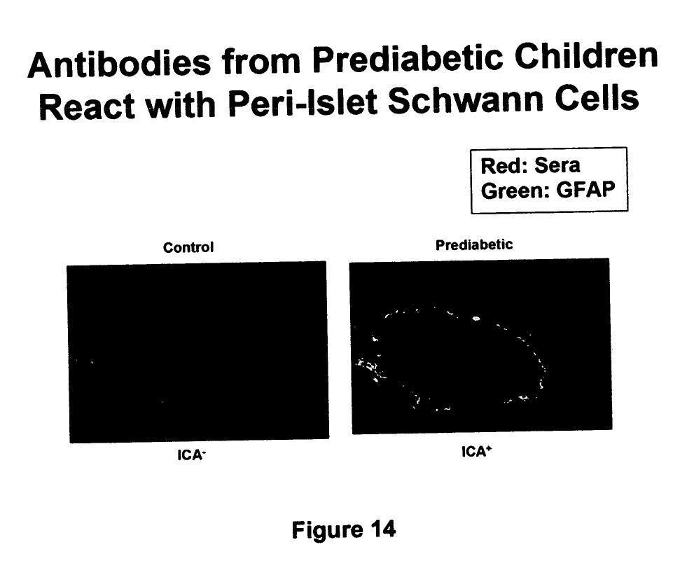

FIG. 14 shows micrographs stained using immunohistochemical techniques evidencing that antibodies from pre-diabetic children react with peri-islet Schwann cells. The left micrograph represents the control sample and the right micrograph represents the pre-diabetic sample. The red stain identifies the sera and the green stain identifies GFAP. The control sample tested ICA-negative and the pre-diabetic ICA⁺. Islet cell cytoplasmic auto-antibodies (ICA) are an early indicator of diabetes. A pre-diabetic patient is defined as individual that is positive for ICA but has not yet shown any outward signs of disease.

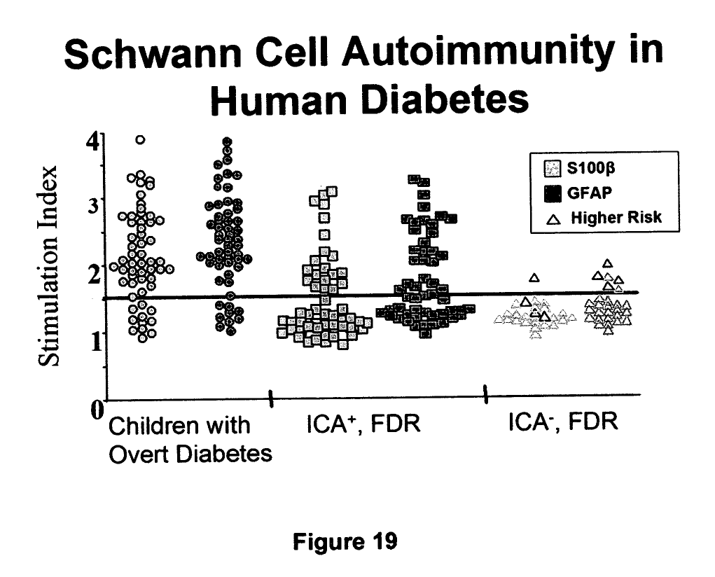

FIG. 19 shows a graph measuring Schwann cell autoimmunity in human diabetes by testing T-cell autoreactivity. The abbreviation “FDR” refers to first degree relative. A first degree relative is defined as an individual who is genetically predisposed to develop Type 1 diabetes. The cumulative risk of developing Type 1 diabetes is 70-80% over 15 years. The yellow color represents the S100β auto-antibody and the green color represents the GFAP auto-antibody. The red triangles identify individuals who may be at higher risk due to the fact that despite testing negative for ICA, they had positive test results to other auto-antigens (data not shown). Subsequent to this study, one of these individuals represented with a red triangle tested positive for ICA. The data shown in FIG. 19 is presented as a stimulation index (cpm antigen stimulated/medium control; background 900-1800 cpm, mean 1,265±215). Positive responses were >3 s.d. above mean OVA responses, P values<0.002 versus healthy controls, Mann Whitney Test.

It is thus concluded that autoimmunity against peri-insular SC is characteristic of human and NOD mouse T1D and thus appears to be a characteristic of the disease in general. Collectively, these observations establish peri-insular Schwann cells as a bona fide autoimmune target in T1D. Autoantibodies are not thought to be mediators of tissue destruction, but rather reflect the immune system's function to remove detritus once tissue destruction occurred. While it is difficult to rule out subtle β-cell damage this early in the pre-diabetes process, the first autoantibody and thus the first tissue destruction in pre-diabetes is the peri-islet SC mantle, i.e. a nervous system tissue. Therefore, antigens of Schwann cell breakdown can be considered the first harbingers of diabetes progression. This conclusion provides not only a new diagnostic element in pre-diabetes (neuronal protein markers), but also an attractive new target for therapeutic, including immunotherapeutic intervention, e.g. modalities such as administration of an immunologically reactive moiety capable of altering the course, progression and/or manifestation of the disease, as a result of interfering with the disease manifestation process at the early stages focused upon by the identification of the disease, e.g. pre-diabetes indicative markers as instantly disclosed, such as by supplying a moiety capable of modifying the pathogenicity of lymphocytes specific for GFAP or other related Schwann cell components.

Therapeutic targets may thus be defined as those moieties which are capable of exerting a modulating force, wherein modulation is defined as an alteration in function inclusive of activity, synthesis, production, and circulating levels. Thus, modulation effects the level or physiological activity of at least one particular disease related biopolymer marker or any compound or biomolecule whose presence, level or activity is linked either directly or indirectly, to an alteration of the presence, level, activity or generic function of the biopolymer marker, and may include pharmaceutical agents, biomolecules that bind to the biopolymer markers, or biomolecules or complexes to which the biopolymer markers bind. The binding of the biopolymer markers and the therapeutic moiety may result in activation (agonist), inhibition (antagonist), or an increase or decrease in activity or production (modulator) of the biopolymer markers or the bound moiety. Examples of such therapeutic moieties include, but are not limited to, antibodies, oligonucleotides, proteins (e.g., receptors), RNA, DNA, enzymes, peptides or small molecules.

With regard to immunotherapeutic moieties, such a moiety would be an effective analogue for a major epitope peptide in a neuronal protein marker which reduces the pathogenicity of key lymphocytes which are specific for the native epitope in the neuronal protein marker. An analogue is defined as having structural similarity but not identity in peptide sequencing able to be recognized by T-cells spontaneously arising and targeting the endogeneous self epitope. A critical function of this analogue is an altered T-cell activation which leads to T-cell anergy or death.

As β-cells have gene expression patterns reminiscent of neuronal cells (F. Atouf, P. Czernichow, R. Scharfmann, J Biol Chem 272, 1929-34 (1997)), it seems conceivable that interactions between peri-islet Schwann cells and intra-islet β-cells have functional interactions typical for peripheral Schwann cells and ‘their’ neurons, with the former maintaining the latter. An autoimmune attack on Schwann cells would then compromise survival of β-cells and possibly their regeneration. This possible axis of interaction has been uncovered by the observations leading to the present invention and deserve renewed attention as a candidate factor in pre-diabetes progression: e.g. β-cells may be victims of collateral damage in a primary autoimmune attack on pancreatic nervous system tissue.

As used herein the term “marker” or “biopolymer marker” are any molecules, typically proteins that pass out from the organ's cells as the cells become damaged or as adaptation occurs. These proteins can be either in the native form or can be any moiety which contains immunologically detectable or immunologically reactive fragments of the protein, resulting, for example, from proteolytic digestion of the protein. When the terms “marker” “biopolymer marker” or “analyte” are used, they are intended to include fragments thereof that can be immunologically detected. By “immunologically detectable” or “immunologically reactive” is meant that the protein fragments contain an epitope that is specifically recognized by a cognate antibody, e.g. the immunologically reactive marker, moiety or fragment has an affinity for a particular entity, e.g. an antibody.

As used herein, the term antibody includes polyclonal and monoclonal antibodies of any isotype (IgA, IgG, IgE, IgD, IgM), or an antigen-binding portion thereof, including but not limited to F(ab) and Fv fragments, single chain antibodies, chimeric antibodies, humanized antibodies, and a Fab expression library.

Antibodies useful as detector and capture antibodies in the present invention may be prepared by standard techniques well known in the art. The antibodies can be used in any type of immunoassay. This includes both the two-site sandwich assay and the single site immunoassay of the non-competitive type, as well as in traditional competitive binding assays.

Particularly preferred, for ease and simplicity of detection, and its quantitative nature, is the sandwich or double antibody assay of which a number of variations exist, all of which are contemplated by the present invention. For example, in a typical sandwich assay, unlabeled antibody is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

The assays may be competitive assays, sandwich assays, and the label may be selected from the group of well-known labels such as radioimmunoassay, fluorescent or chemiluminescence immunoassay, or immunoPCR technology. Extensive discussion of the known immunoassay techniques is not required here since these are known to those of skilled in the art. See Takahashi et al. (Clin Chem 1999;45(8):1307) for S100B assay.

The assays contemplated within the scope of the instant invention may test for a single neuronal protein marker or test for multiple neuronal protein markers simultaneously.

Although not wishing to be limited to any particular embodiment, the panel format exemplified herein is known and is commercially available. The panel format is similar to a format currently being used in association with pregnancy testing and is commercially available under the trade-mark BIOSIGN. Any assay device or method in accordance with the objectives of the instant invention is contemplated for use with one or more bodily fluids, said bodily fluids being selected from the group consisting of blood, blood components, urine, saliva, lymph and cerebrospinal fluid.

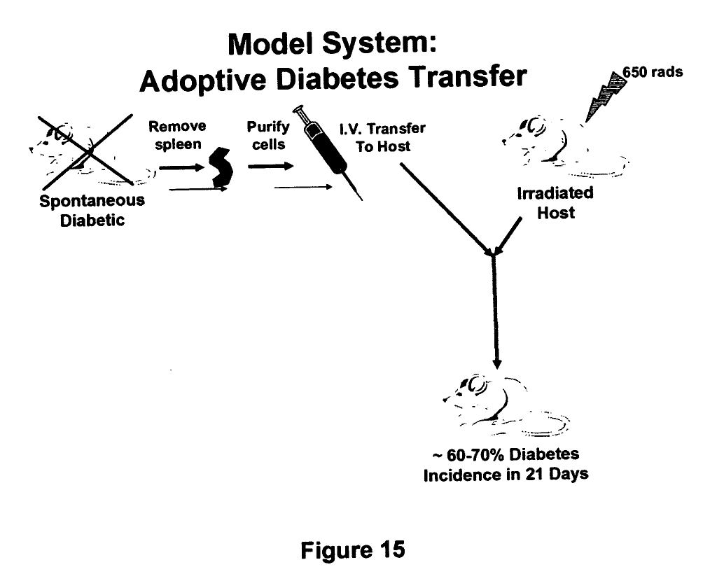

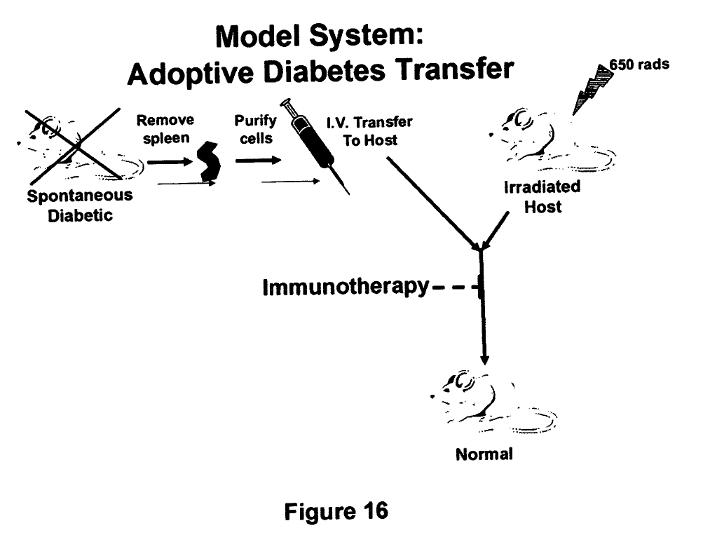

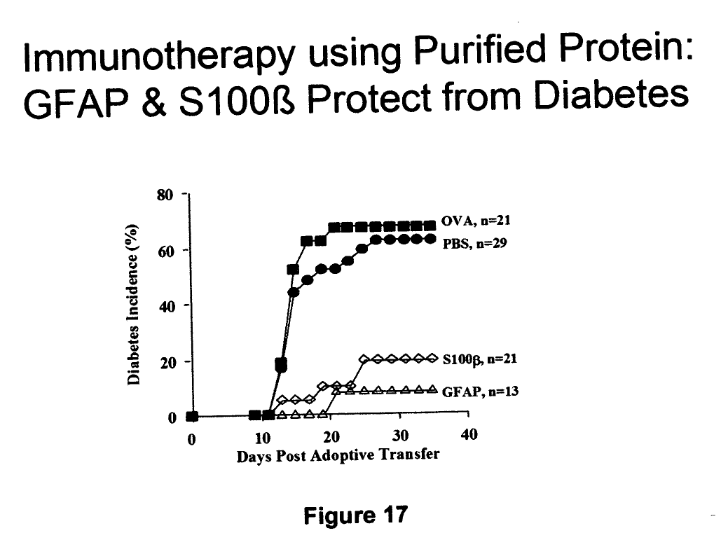

The discovery that nervous system auto-immunity occurs early in Type 1 diabetes allows for earlier diagnosis and therefore earlier intervention. Thus, it may be possible to inhibit disease progression before the insulin-producing cells are destroyed. The instant inventors contemplate two immunotherapeutic approaches to inhibition of disease progression; administration of purified proteins and administration of protein epitopes that are specific for reactive T-cells. FIG. 15 shows a schematic view of the model system for adoptive diabetes transfer used to test immunotherapeutic approaches to disease inhibition. The spleen of a diabetic mouse is removed, the cells purified and transferred by IV to an irradiated host mouse (650 rads). Within 21 days, 60-70% of these host mice developed diabetes. However, as illustrated in FIG. 16, when immunotherapy is administered to the irradiated host mice; the mice remain healthy (normal). The immunotherapeutic approach in this experiment consisted of the administration of purified GFAP and S100β. This administration of purified protein protects the mice from diabetes. FIG. 17 shows a graph charting the incidence of diabetes in the treated mice (%) against days post adoptive transfer. PBS and OVA were administered as controls. FIG. 17 evidences that less than 20% of the mice treated with S100β or GFAP developed diabetes when tested over a 40 day period.

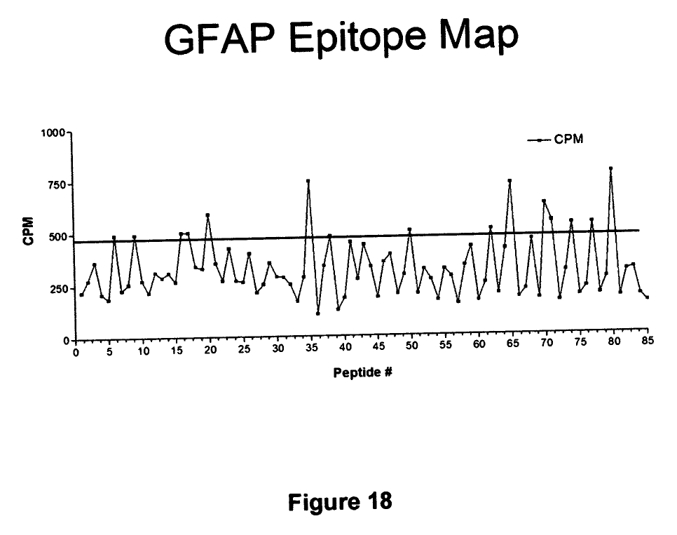

Currently, the instant inventors are working on the identification of epitopes specific for reactive T-cells. The administration of these epitopes should reduce the pathogenicity of the T-cells by inducing an altered T-cell activation which leads to T-cell anergy or death. In this experiment, epitope mapping is conducted with human diabetic samples. FIG. 18 illustrates an epitope map for GFAP.

In summary, the instant invention provides markers and methods useful for early diagnosis of pre-Type-1 diabetes based on the discovery that nervous system proteins play a role in the early stage autoimmunity of diabetic pathology.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The various biomolecules, e.g. antibodies, markers, oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for diagnosing pre-Type 1 diabetes comprising the steps of:
(a) obtaining a sample of a bodily fluid from subjects within an at-risk population and;
(b) analyzing said sample for a clinically relevant presence of at least one neuronal tissue marker wherein said clinically relevant presence of at least one neuronal tissue marker is diagnostic for pre-Type 1 diabetes.

2. The method in accordance with claim 1 wherein said at-risk population is a target population.

3. The method in accordance with claim 1 wherein said at least one neuronal tissue marker is selected from the group consisting of GFAP (glial fibrillary acidic protein), NSE (neuron specific enolase), S100β, CNPase (2′, 3′-cyclic nucleotide 3′-phosphodiesterase) and fragments thereof.

4. The method in accordance with claim 2 wherein said at least one neuronal tissue marker is selected from the group consisting of GFAP, NSE, S100β, CNPase and fragments thereof.

5. The method in accordance with claim 1 wherein said sample of a bodily fluid is selected from the group consisting of blood, blood products, urine, saliva, cerebrospinal fluid and lymph.

6. The method in accordance with claim 2 wherein said sample of a bodily fluid is selected from the group consisting of blood, blood products, urine, saliva, cerebrospinal fluid and lymph.

7. A kit for diagnosing and staging pre-Type I diabetes comprising:
reagents for detecting GFAP, NSE, S100β, CNPase and fragments thereof; wherein a clinically relevant presence of at least one neuronal tissue marker selected from the group consisting of GFAP, NSE, S100β, CNPase and fragments thereof is determined; whereby a diagnosis of pre-Type I diabetes is ascertained and disease staging is determined.

8. A method for diagnosing pre-Type 1 diabetes comprising the steps of:
(a) obtaining a sample of a bodily fluid from subjects within an at-risk population and;
(b) analyzing said sample for a clinically relevant presence of at least one autoantibody for a neuronal tissue marker wherein said clinically relevant presence of at least one autoantibody for a neuronal tissue marker is diagnostic for pre-Type 1 diabetes.

9. The method in accordance with claim 8 wherein said at-risk population is a target population.

10. The method in accordance with claim 8 wherein said at least one autoantibody for a neuronal tissue marker is selected from the group consisting of autoantibodies for GFAP, NSE, S100β, CNPase and fragments thereof.

11. The method in accordance with claim 9 wherein said at least one autoantibody for a neuronal tissue marker is selected from the group consisting of autoantibodies for GFAP, NSE, S100β, CNPase and fragments thereof.

12. The method in accordance with claim 8 wherein said sample of a bodily fluid is selected from the group consisting of blood, blood products, urine, saliva, cerebrospinal fluid and lymph.

13. The method in accordance with claim 9 wherein said sample of a bodily fluid is selected from the group consisting of blood, blood products, urine, saliva, cerebrospinal fluid and lymph.

14. A kit for diagnosing and staging pre-Type 1 diabetes comprising:
reagents for detecting autoantibodies for GFAP, NSE, S100β, CNPase and fragments thereof; wherein a clinically relevant presence of at least one autoantibody selected from the group consisting of autoantibodies for GFAP, NSE, S100β, CNPase and fragments thereof is determined; whereby a diagnosis of pre-Type I diabetes is ascertained and disease staging is determined.

15. A method for diagnosing pre-Type I diabetes comprising the steps of:
(a) obtaining a sample of a bodily fluid from a subject within an at-risk population;
(b) analyzing said sample for a clinically relevant presence of at least one neuronal tissue marker selected from the group consisting of GFAP, NSE, S100β, CNPase and fragments thereof; and
(c) analyzing said sample for a clinically relevant presence of at least one autoantibody for a neuronal tissue marker selected from the group consisting of autoantibodies for GFAP, NSE, S100β, CNPase and fragments thereof; wherein a clinically relevant presence of said at least one neuronal tissue marker and a clinically relevant presence of said at least one autoantibody for a neuronal tissue marker is determined; whereby a diagnosis of pre-Type 1 diabetes is ascertained and disease staging is determined.

16. The method of claim 15 wherein said at least one neuronal tissue marker is GFAP or a fragment thereof and said at least one autoantibody for a neuronal tissue marker is the corresponding autoantibody for GFAP or a fragment thereof.

17. The method of claim 15 wherein said at least one neuronal tissue marker is NSE or a fragment thereof and said at least one autoantibody for a neuronal tissue marker is the corresponding autoantibody for NSE or a fragment thereof.

18. The method of claim 15 wherein said at least one neuronal tissue marker is S100β or a fragment thereof and said at least one autoantibody for a neuronal tissue marker is the corresponding autoantibody for S100β or a fragment thereof.

19. The method of claim 15 wherein said at least one neuronal tissue marker is CNPase or a fragment thereof and said at least one autoantibody for a neuronal tissue marker is the corresponding autoantibody for CNPase or a fragment thereof.

20. A kit for diagnosing and staging pre-Type I diabetes comprising:
reagents for detecting GFAP, NSE, S100β, CNPase and fragments thereof; and
reagents for detecting autoantibodies for GFAP, NSE, S100β, CNPase and fragments thereof; wherein a clinically relevant presence of at least one neuronal tissue marker selected from the group consisting of GFAP, NSE, S100β, CNPase and fragments thereof, and a clinically relevant presence of at least one autoantibody selected from the group consisting of autoantibodies for GFAP, NSE, S100β, CNPase and fragments thereof is determined; whereby a diagnosis of pre-Type I diabetes is ascertained and disease staging is determined.

21. A method for diagnosing pre-Type 1 diabetes comprising the steps of:
(a) obtaining a sample of a bodily fluid from subjects within an at-risk population and;
(b) analyzing said sample for a clinically relevant presence of GAD65 or a fragment thereof and a clinically relevant presence of at least one additional neuronal tissue marker or a fragment thereof, wherein said clinically relevant presence of said GAD65 or fragment thereof and said clinically relevant presence of said at least one additional neuronal tissue marker or a fragment thereof is determined; whereby a diagnosis of pre-Type 1 diabetes is ascertained and disease staging determined.

22. The method in accordance with claim 21 wherein said at-risk population is a target population.

23. The method in accordance with claim 21 wherein said at least one additional neuronal tissue marker is selected from the group consisting of GFAP, NSE, S100β, CNPase and fragments thereof.

24. The method in accordance with claim 21 wherein said sample of a bodily fluid is selected from the group consisting of blood, blood products, urine, saliva, cerebrospinal fluid and lymph.

25. The method in accordance with claim 22 wherein said at least one additional neuronal tissue marker is selected from the group consisting of GFAP, NSE, S100β, CNPase and fragments thereof.

26. The method in accordance with claim 22 wherein said sample of a bodily fluid is selected from the group consisting of blood, blood products, urine, saliva, cerebrospinal fluid and lymph.

27. A kit for diagnosing and staging pre-Type I diabetes comprising:
reagents for detecting GAD65, GFAP, NSE, S100β, CNPase and fragments thereof;
wherein a clinically relevant presence of GAD65 or a fragment thereof and a clinically relevant presence of at least one additional neuronal tissue marker selected from the group consisting of GFAP, NSE, S100β, CNPase and fragments thereof is determined;
whereby a diagnosis of pre-Type I diabetes is ascertained and disease staging is determined.

28. A method for diagnosing pre-Type 1 diabetes comprising the steps of:
(a) obtaining a sample of a bodily fluid from subjects within an at-risk population and;
(b) analyzing said sample for a clinically relevant presence of an autoantibody for GAD65 or a fragment thereof and a clinically relevant presence of at least one neuronal tissue marker or a fragment thereof, wherein said clinically relevant presence of said autoantibody for GAD65 or fragment thereof and said clinically relevant presence of said at least one neuronal tissue marker or fragment thereof is diagnostic for pre-Type 1 diabetes.

29. The method in accordance with claim 28 wherein said at-risk population is a target population.

30. The method in accordance with claim 28 wherein said at least one neuronal tissue marker is selected from the group consisting of GFAP, NSE, S100β, CNPase and fragments thereof.

31. The method in accordance with claim 28 wherein said sample of a bodily fluid is selected from the group consisting of blood, blood products, urine, saliva, cerebrospinal fluid and lymph.

32. The method in accordance with claim 29 wherein said at least one neuronal tissue marker is selected from the group consisting of GFAP, NSE, S100β, CNPase and fragments thereof.

33. The method in accordance with claim 29 wherein said sample of a bodily fluid is selected from the group consisting of blood, blood products, urine, saliva, cerebrospinal fluid and lymph.

34. A kit for diagnosing and staging pre-Type I diabetes comprising:
reagents for detecting an autoantibody for GAD65 and fragments thereof; and
reagents for detecting GFAP, NSE, S100β, CNPase and fragments thereof;
wherein a clinically relevant presence of an autoantibody for GAD65 or a fragment thereof and a clinically relevant presence of at least one neuronal tissue marker selected from the group consisting of GFAP, NSE, S100β, CNPase and fragments thereof is determined;
whereby a diagnosis of pre-Type I diabetes is ascertained and disease staging is determined.

35. A method for diagnosing pre-Type 1 diabetes comprising the steps of:
(a) obtaining a sample of a bodily fluid from subjects within an at-risk population and;
(b) analyzing said sample for a clinically relevant presence of GAD65 or a fragment thereof and a clinically relevant presence of at least one autoantibody for a neuronal tissue marker or a fragment thereof, wherein said clinically relevant presence of said GAD65 or fragment thereof and said clinically relevant presence of said at least one autoantibody for a neuronal tissue marker or fragment thereof is diagnostic for pre-Type 1 diabetes.

36. The method in accordance with claim 35 wherein said at-risk population is a target population.

37. The method in accordance with claim 35 wherein said at least one autoantibody for a neuronal tissue marker is selected from the group consisting of autoantibodies for GFAP, NSE, S100β, CNPase and fragments thereof.

38. The method in accordance with claim 35 wherein said sample of a bodily fluid is selected from the group consisting of blood, blood products, urine, saliva, cerebrospinal fluid and lymph.

39. The method in accordance with claim 36 wherein said at least one autoantibody for a neuronal tissue marker is selected from the group consisting of autoantibodies for GFAP, NSE, S100β, CNPase and fragments thereof.

40. The method in accordance with claim 36 wherein said sample of a bodily fluid is selected from the group consisting of blood, blood products, urine, saliva, cerebrospinal fluid and lymph.

41. A kit for diagnosing and staging pre-Type I diabetes comprising:
reagents for detecting GAD65 and fragments thereof; and
reagents for detecting an autoantibody for GFAP, NSE, S100β, CNPase and fragments thereof;
wherein a clinically relevant presence of GAD65 or fragments thereof and a clinically relevant presence of at least one autoantibody for a neuronal tissue marker selected from the group consisting of autoantibodies for GFAP, NSE, S100β, CNPase and fragments thereof is determined;
whereby a diagnosis of pre-Type I diabetes is ascertained and disease staging is determined.

42. A method for diagnosing pre-Type 1 diabetes comprising the steps of:
(a) obtaining a sample of a bodily fluid from subjects within an at-risk population and;
(b) analyzing said sample for a clinically relevant presence of an autoantibody for GAD65 or a fragment thereof and a clinically relevant presence of at least one additional autoantibody for a neuronal tissue marker or a fragment thereof, wherein said clinically relevant presence of said autoantibody for GAD65 or a fragment thereof and said clinically relevant presence of said at least one additional autoantibody for a neuronal tissue marker or fragment thereof is diagnostic for pre-Type 1 diabetes.

43. The method in accordance with claim 42 wherein said at-risk population is a target population.

44. The method in accordance with claim 42 wherein said at least one additional autoantibody for a neuronal tissue marker is selected from the group consisting of autoantibodies for GFAP, NSE, S100β, CNPase and fragments thereof.

45. The method in accordance with claim 42 wherein said sample of a bodily fluid is selected from the group consisting of blood, blood products, urine, saliva, cerebrospinal fluid and lymph.

46. The method in accordance with claim 43 wherein said at least one additional autoantibody for a neuronal tissue marker is selected from the group consisting of autoantibodies for GFAP, NSE, S100β, CNPase and fragments thereof.

47. The method in accordance with claim 43 wherein said sample of a bodily fluid is selected from the group consisting of blood, blood products, urine, saliva, cerebrospinal fluid and lymph.

48. A kit for diagnosing and staging pre-Type I diabetes comprising:
reagents for detecting an autoantibody for GAD65 and fragments thereof; and
reagents for detecting an autoantibody for GFAP, NSE, S100β, CNPase and fragments thereof;
wherein a clinically relevant presence of an autoantibody for GAD65 or fragments thereof and a clinically relevant presence of at least one additional autoantibody for a neuronal tissue marker selected from the group consisting of autoantibodies for GFAP, NSE, S100β, CNPase or fragments thereof is determined;
whereby a diagnosis of pre-Type I diabetes is ascertained and disease staging is determined.

49. A method for diagnosing pre-Type 1 diabetes comprising the steps of:
(a) obtaining a sample of a bodily fluid from subjects within an at-risk population and;
(b) analyzing said sample for a clinically relevant presence of GAD65 or a fragment thereof, a clinically relevant presence of an autoantibody for GAD65 or a fragment thereof and a clinically relevant presence of at least one additional neuronal tissue marker or a fragment thereof, wherein said clinically relevant presence of said GAD65 or a fragment thereof, said clinically relevant presence of an autoantibody for GAD65 or a fragment thereof and said clinically relevant presence of said at least one additional neuronal tissue marker or fragment thereof is diagnostic for pre-Type 1 diabetes.

50. The method in accordance with claim 49 wherein said at-risk population is a target population.

51. The method in accordance with claim 49 wherein said at least one additional neuronal tissue marker is selected from the group consisting of GFAP, NSE, S100β, CNPase and fragments thereof.

52. The method in accordance with claim 49 wherein said sample of a bodily fluid is selected from the group consisting of blood, blood products, urine, saliva, cerebrospinal fluid and lymph.

53. The method in accordance with claim 50 wherein said at least one additional neuronal tissue marker is selected from the group consisting of GFAP, NSE, S100β, CNPase and fragments thereof.

54. The method in accordance with claim 50 wherein said sample of a bodily fluid is selected from the group consisting of blood, blood products, urine, saliva, cerebrospinal fluid and lymph.

55. A kit for diagnosing and staging pre-Type I diabetes comprising:
reagents for detecting GAD65 and fragments thereof;
reagents for detecting an autoantibody for GAD65 and fragments thereof; and
reagents for detecting GFAP, NSE, S100β, CNPase and fragments thereof;
wherein a clinically relevant presence of GAD65 or fragments thereof, a clinically relevant presence of an autoantibody for GAD65 or fragments thereof and a clinically relevant presence of at least one additional neuronal tissue marker selected from the group consisting of GFAP, NSE, S100β, CNPase and fragments thereof is determined;
whereby a diagnosis of pre-Type I diabetes is ascertained and disease staging is determined.

56. A method for diagnosing pre-Type 1 diabetes comprising the steps of:
(a) obtaining a sample of a bodily fluid from subjects within an at-risk population and;
(b) analyzing said sample for a clinically relevant presence of GAD65 or a fragment thereof, a clinically relevant presence of an autoantibody for GAD65 or a fragment thereof and a clinically relevant presence of at least one additional autoantibody for a neuronal tissue marker or a fragment thereof, wherein said clinically relevant presence of said GAD65 or a fragment thereof, said clinically relevant presence of an autoantibody for GAD65 or a fragment thereof and said clinically relevant presence of said at least one additional autoantibody for a neuronal tissue marker or fragment thereof is diagnostic for pre-Type 1 diabetes.

57. The method in accordance with claim 56 wherein said at-risk population is a target population.

58. The method in accordance with claim 56 wherein said at least one additional autoantibody for a neuronal tissue marker is selected from the group consisting of autoantibodies for GFAP, NSE, S100β, CNPase and fragments thereof.

59. The method in accordance with claim 56 wherein said sample of a bodily fluid is selected from the group consisting of blood, blood products, urine, saliva, cerebrospinal fluid and lymph.

60. The method in accordance with claim 57 wherein said at least one additional autoantibody for a neuronal tissue marker is selected from the group consisting of autoantibodies for GFAP, NSE, S100β, CNPase and fragments thereof.

61. The method in accordance with claim 57 wherein said sample of a bodily fluid is selected from the group consisting of blood, blood products, urine, saliva, cerebrospinal fluid and lymph.

62. A kit for diagnosing and staging pre-Type I diabetes comprising:
reagents for detecting GAD65 and fragments thereof;
reagents for detecting an autoantibody for GAD65 and fragments thereof; and
reagents for detecting an autoantibody for GFAP, NSE, S100β, CNPase and fragments thereof;
wherein a clinically relevant presence of GAD65 or fragments thereof, a clinically relevant presence of an autoantibody for GAD65 or fragments thereof and a clinically relevant presence of at least one additional autoantibody for a neuronal tissue marker selected from the group consisting of autoantibodies for GFAP, NSE, S100β, CNPase and fragments thereof is determined;
whereby a diagnosis of pre-Type I diabetes is ascertained and disease staging is determined.

63. A method for diagnosing pre-Type 1 diabetes comprising the steps of:
(a) obtaining a sample of a bodily fluid from subjects within an at-risk population and;
(b) analyzing said sample for a clinically relevant presence of GAD65 or a fragment thereof, a clinically relevant presence of at least one additional neuronal tissue marker or a fragment thereof and a clinically relevant presence of at least one autoantibody for a neuronal tissue marker or a fragment thereof, wherein said clinically relevant presence of said GAD65 or fragment thereof, said clinically relevant presence of said at least one additional neuronal tissue marker or fragment thereof and said clinically relevant presence of said at least one autoantibody for a neuronal tissue marker or fragment thereof is diagnostic for pre-Type 1 diabetes.

64. The method in accordance with claim 63 wherein said at-risk population is a target population.

65. The method in accordance with claim 63 wherein said at least one additional neuronal tissue marker is selected from the group consisting of GFAP, NSE, S100β, CNPase and fragments thereof.

66. The method in accordance with claim 63 wherein said at least one autoantibody for a neuronal tissue marker is selected from the group consisting of autoantibodies for GFAP, NSE, S100β, CNPase and fragments thereof.

67. The method in accordance with claim 63 wherein said sample of a bodily fluid is selected from the group consisting of blood, blood products, urine, saliva, cerebrospinal fluid and lymph.

68. The method in accordance with claim 64 wherein said at least one additional neuronal tissue marker is selected from the group consisting of GFAP, NSE, S100β, CNPase and fragments thereof.

69. The method in accordance with claim 64 wherein said at least one autoantibody for a neuronal tissue marker is selected from the group consisting of autoantibodies for GFAP, NSE, S100β, CNPase and fragments thereof.

70. The method in accordance with claim 64 wherein said sample of a bodily fluid is selected from the group consisting of blood, blood products, urine, saliva, cerebrospinal fluid and lymph.

71. A kit for diagnosing and staging pre-Type I diabetes comprising:
reagents for detecting GAD65 and fragments thereof;
reagents for detecting GFAP, NSE, S100β, CNPase and fragments thereof; and
reagents for detecting autoantibodies for GFAP, NSE, S100β, CNPase and fragments thereof;
wherein a clinically relevant presence of GAD65 or fragments thereof, a clinically relevant presence of at least one additional neuronal tissue marker selected from the group consisting of GFAP, NSE, S100β, CNPase and fragments thereof and a clinically relevant presence of at least one autoantibody for a neuronal tissue marker selected from the group consisting of autoantibodies for GFAP, NSE, S100β, CNPase and fragments thereof is determined;
whereby a diagnosis of pre-Type I diabetes is ascertained and disease staging is determined.

72. A method for diagnosing pre-Type 1 diabetes comprising the steps of:
(a) obtaining a sample of a bodily fluid from subjects within an at-risk population and;
(b) analyzing said sample for a clinically relevant presence of at least one neuronal tissue marker or a fragment thereof, a clinically relevant presence of at least one autoantibody for a neuronal tissue marker or a fragment thereof and a clinically relevant presence of an autoantibody for GAD65 or a fragment thereof, wherein said clinically relevant presence of at least one neuronal tissue marker or fragment thereof, said clinically relevant presence of said at least one autoantibody for a neuronal tissue marker or fragment thereof and said clinically relevant presence of an autoantibody for GAD65 or a fragment thereof is diagnostic for pre-Type 1 diabetes.

73. The method in accordance with claim 72 wherein said at-risk population is a target population.

74. The method in accordance with claim 72 wherein said at least one neuronal tissue marker is selected from the group consisting of GFAP, NSE, S100β, CNPase and fragments thereof.

75. The method in accordance with claim 72 wherein said at least one autoantibody for a neuronal tissue marker is selected from the group consisting of autoantibodies for GFAP, NSE, S100β, CNPase and fragments thereof.

76. The method in accordance with claim 72 wherein said sample of a bodily fluid is selected from the group consisting of blood, blood products, urine, saliva, cerebrospinal fluid and lymph.

77. The method in accordance with claim 73 wherein said at least one neuronal tissue marker is selected from the group consisting of GFAP, NSE, S100β, CNPase and fragments thereof.

78. The method in accordance with claim 73 wherein said at least one autoantibody for a neuronal tissue marker is selected from the group consisting of autoantibodies for GFAP, NSE, S100β, CNPase and fragments thereof.

79. The method in accordance with claim 73 wherein said sample of a bodily fluid is selected from the group consisting of blood, blood products, urine, saliva, cerebrospinal fluid and lymph.

80. A kit for diagnosing and staging pre-Type I diabetes comprising:
reagents for detecting GFAP, NSE, S100β, CNPase and fragments thereof;
reagents for detecting autoantibodies for GFAP, NSE, S100β, CNPase and fragments thereof; and
reagents for detecting an autoantibody for GAD65 and fragments thereof;
wherein a clinically relevant presence of at least one neuronal tissue marker selected from the group consisting of GFAP, NSE, S100β, CNPase and fragments thereof, a clinically relevant presence of at least one autoantibody for a neuronal tissue marker selected from the group consisting of autoantibodies for GFAP, NSE, S100β, CNPase and fragments thereof and a clinically relevant presence of an autoantibody for GAD65 or fragments thereof is determined;
whereby a diagnosis of pre-Type I diabetes is ascertained and disease staging is determined.

81. A method for diagnosing pre-Type 1 diabetes comprising the steps of:
(a) obtaining a sample of a bodily fluid from subjects within an at-risk population and;
(b) analyzing said sample for a clinically relevant presence of GAD65 or a fragment thereof, a clinically relevant presence of at least one additional neuronal tissue marker or a fragment thereof, a clinically relevant presence of an autoantibody for GAD65 or a fragment thereof and a clinically relevant presence of at least one additional autoantibody for a neuronal tissue marker or a fragment thereof, wherein said clinically relevant presence of said GAD65 or fragment thereof, said clinically relevant presence of said at least one additional neuronal tissue marker or fragment thereof, said clinically relevant presence of said autoantibody for GAD65 or a fragment thereof and said clinically relevant presence of said at least one additional autoantibody for a neuronal tissue marker or fragment thereof is diagnostic for pre-Type 1 diabetes.

82. The method in accordance with claim 81 wherein said at-risk population is a target population.

83. The method in accordance with claim 81 wherein said at least one additional neuronal tissue marker is selected from the group consisting of GFAP, NSE, S100β, CNPase and fragments thereof.

84. The method in accordance with claim 81 wherein said at least one additional autoantibody for a neuronal tissue marker is selected from the group consisting of autoantibodies for GFAP, NSE, S100β, CNPase and fragments thereof.

85. The method in accordance with claim 81 wherein said sample of a bodily fluid is selected from the group consisting of blood, blood products, urine, saliva, cerebrospinal fluid and lymph.

86. The method in accordance with claim 82 wherein said at least one additional neuronal tissue marker is selected from the group consisting of GFAP, NSE, S100β, CNPase and fragments thereof.

87. The method in accordance with claim 82 wherein said at least one autoantibody for a neuronal tissue marker is selected from the group consisting of autoantibodies for GFAP, NSE, S100β, CNPase and fragments thereof.

88. The method in accordance with claim 82 wherein said sample of a bodily fluid is selected from the group consisting of blood, blood products, urine, saliva, cerebrospinal fluid and lymph.

89. A kit for diagnosing and staging pre-Type I diabetes comprising:
reagents for detecting GAD65 and fragments thereof;
reagents for detecting GFAP, NSE, S100β, CNPase and fragments thereof;
reagents for detecting autoantibodies for GFAP, NSE, S100β, CNPase and fragments thereof; and
reagents for detecting an autoantibody for GAD65 and fragments thereof;
wherein a clinically relevant presence of GAD65 or fragments thereof, a clinically relevant presence of at least one additional neuronal tissue marker selected from the group consisting of GFAP, NSE, S100β, CNPase and fragments thereof, a clinically relevant presence of an autoantibody for GAD65 or fragments thereof and a clinically relevant presence of at least one additional autoantibody for a neuronal tissue marker selected from the group consisting of autoantibodies for GFAP, NSE, S100β, CNPase and fragments thereof is determined;
whereby a diagnosis of pre-Type I diabetes is ascertained and disease staging is determined.

90. The method in accordance with claim 63 wherein said at least one additional neuronal tissue marker is selected from the group consisting of GFAP, NSE, S100β, CNPase and fragments thereof and wherein said at least one autoantibody for a neuronal tissue marker is selected from the group consisting of autoantibodies for GFAP, NSE, S100β, CNPase and fragments thereof.

91. The method in accordance with claim 90 wherein said sample of a bodily fluid is selected from the group consisting of blood, blood products, urine, saliva, cerebrospinal fluid and lymph.

92. The method in accordance with claim 64 wherein said at least one additional neuronal tissue marker is selected from the group consisting of GFAP, NSE, S100β, CNPase and fragments thereof and wherein said at least one autoantibody for a neuronal tissue marker is selected from the group consisting of autoantibodies for GFAP, NSE, S100β, CNPase and fragments thereof.

93. The method in accordance with claim 92 wherein said sample of a bodily fluid is selected from the group consisting of blood, blood products, urine, saliva, cerebrospinal fluid and lymph.

94. The method in accordance with claim 72 wherein said at least one neuronal tissue marker is selected from the group consisting of GFAP, NSE, S100β, CNPase and fragments thereof and wherein said at least one autoantibody for a neuronal tissue marker is selected from the group consisting of autoantibodies for GFAP, NSE, S100β, CNPase and fragments thereof.

95. The method in accordance with claim 94 wherein said sample of a bodily fluid is selected from the group consisting of blood, blood products, urine, saliva, cerebrospinal fluid and lymph.

96. The method in accordance with claim 73 wherein said at least one neuronal tissue marker is selected from the group consisting of GFAP, NSE, S100β, CNPase and fragments thereof and wherein said at least one autoantibody for a neuronal tissue marker is selected from the group consisting of autoantibodies for GFAP, NSE, S100β, CNPase and fragments thereof.

97. The method in accordance with claim 96 wherein said sample of a bodily fluid is selected from the group consisting of blood, blood products, urine, saliva, cerebrospinal fluid and lymph.

98. The method in accordance with claim 81 wherein said at least one additional neuronal tissue marker is selected from the group consisting of GFAP, NSE, S100β, CNPase and fragments thereof and wherein said at least one additional autoantibody for a neuronal tissue marker is selected from the group consisting of autoantibodies for GFAP, NSE, S100β, CNPase and fragments thereof.

99. The method in accordance with claim 98 wherein said sample of a bodily fluid is selected from the group consisting of blood, blood products, urine, saliva, cerebrospinal fluid and lymph.

100. The method in accordance with claim 82 wherein said at least one additional neuronal tissue marker is selected from the group consisting of GFAP, NSE, S100β, CNPase and fragments thereof and wherein said at least one additional autoantibody for a neuronal tissue marker is selected from the group consisting of autoantibodies for GFAP, NSE, S100β, CNPase and fragments thereof.

101. The method in accordance with claim 100 wherein said sample of a bodily fluid is selected from the group consisting of blood, blood products, urine, saliva, cerebrospinal fluid and lymph.

102. The method in accordance with claim 2 wherein said target population comprises first degree relatives (FDR) of patients having Type-1 diabetes; said FDR ranging from 3-40 years in age.

103. The method in accordance with claim 102 wherein said at least one neuronal tissue marker is selected from the group consisting of GFAP, NSE, S100β, CNPase and fragments thereof.

104. The method in accordance with claim 102 wherein said sample of a bodily fluid is selected from the group consisting of blood, blood products, urine, saliva cerebrospinal fluid and lymph.

105. The method in accordance with claim 9 wherein said target population comprises first degree relatives (FDR) of patients having Type-1 diabetes; said FDR ranging from 3-40 years in age.

106. The method in accordance with claim 105 wherein said at least one autoantibody for a neuronal tissue marker is selected from the group consisting of autoantibodies for GFAP, NSE, S100β, CNPase and fragments thereof.

107. The method in accordance with claim 105 wherein said sample of a bodily fluid is selected from the group consisting of blood, blood products, urine, saliva cerebrospinal fluid and lymph.

108. The method in accordance with claim 22 wherein said target population comprises first degree relatives (FDR) of patients having Type-1 diabetes; said FDR ranging from 3-40 years in age.

109. The method in accordance with claim 108 wherein said at least one additional neuronal tissue marker is selected from the group consisting of GFAP, NSE, S100β, CNPase and fragments thereof.

110. The method in accordance with claim 108 wherein said sample of a bodily fluid is selected from the group consisting of blood, blood products, urine, saliva cerebrospinal fluid and lymph.

111. The method in accordance with claim 29 wherein said target population comprises first degree relatives (FDR) of patients having Type-1 diabetes; said FDR ranging from 3-40 years in age.

112. The method in accordance with claim 111 wherein said at least one neuronal tissue marker is selected from the group consisting of GFAP, NSE, S100β, CNPase and fragments thereof.

113. The method in accordance with claim 111 wherein said sample of a bodily fluid is selected from the group consisting of blood, blood products, urine, saliva cerebrospinal fluid and lymph.

114. The method in accordance with claim 36 wherein said target population comprises first degree relatives (FDR) of patients having Type-1 diabetes; said FDR ranging from 3-40 years in age.

115. The method in accordance with claim 114 wherein said at least one autoantibody for a neuronal tissue marker is selected from the group consisting of autoantibodies for GFAP, NSE, S100β, CNPase and fragments thereof.

116. The method in accordance with claim 114 wherein said sample of a bodily fluid is selected from the group consisting of blood, blood products, urine, saliva cerebrospinal fluid and lymph.

117. The method in accordance with claim 43 wherein said target population comprises first degree relatives (FDR) of patients having Type-1 diabetes; said FDR ranging from 3-40 years in age.

118. The method in accordance with claim 117 wherein said at least one additional autoantibody for a neuronal tissue marker is selected from the group consisting of autoantibodies for GFAP, NSE, S100β, CNPase and fragments thereof.

119. The method in accordance with claim 117 wherein said sample of a bodily fluid is selected from the group consisting of blood, blood products, urine, saliva cerebrospinal fluid and lymph.

120. The method in accordance with claim 50 wherein said target population comprises first degree relatives (FDR) of patients having Type-1 diabetes; said FDR ranging from 3-40 years in age.

121. The method in accordance with claim 120 wherein said at least one additional neuronal tissue marker is selected from the group consisting of GFAP, NSE, S100β, CNPase and fragments thereof.

122. The method in accordance with claim 120 wherein said sample of a bodily fluid is selected from the group consisting of blood, blood products, urine, saliva cerebrospinal fluid and lymph.

123. The method in accordance with claim 57 wherein said target population comprises first degree relatives (FDR) of patients having Type-1 diabetes; said FDR ranging from 3-40 years in age.

124. The method in accordance with claim 123 wherein said at least one additional autoantibody for a neuronal tissue marker is selected from the group consisting of autoantibodies for GFAP, NSE, S100β, CNPase and fragments thereof.

125. The method in accordance with claim 123 wherein said sample of a bodily fluid is selected from the group consisting of blood, blood products, urine, saliva cerebrospinal fluid and lymph.

126. The method in accordance with claim 64 wherein said target population comprises first degree relatives (FDR) of patients having Type-1 diabetes; said FDR ranging from 3-40 years in age.

127. The method in accordance with claim 126 wherein said at least one additional neuronal tissue marker is selected from the group consisting of GFAP, NSE, S100β, CNPase and fragments thereof.

128. The method in accordance with claim 126 wherein said sample of a bodily fluid is selected from the group consisting of blood, blood products, urine, saliva cerebrospinal fluid and lymph.

129. The method in accordance with claim 73 wherein said target population comprises first degree relatives (FDR) of patients having Type-1 diabetes; said FDR ranging from 3-40 years in age.

130. The method in accordance with claim 129 wherein said at least one neuronal tissue marker is selected from the group consisting of GFAP, NSE, S100β, CNPase and fragments thereof.

131. The method in accordance with claim 129 wherein said at least one autoantibody for a neuronal tissue marker is selected from the group consisting of autoantibodies for GFAP, NSE, S100β, CNPase and fragments thereof.

132. The method in accordance with claim 129 wherein said sample of a bodily fluid is selected from the group consisting of blood, blood products, urine, saliva cerebrospinal fluid and lymph.

133. The method in accordance with claim 82 wherein said target population comprises first degree relatives (FDR) of patients having Type-1 diabetes; said FDR ranging from 3-40 years in age.

134. The method in accordance with claim 133 wherein said at least one additional neuronal tissue marker is selected from the group consisting of GFAP, NSE, S100β, CNPase and fragments thereof.

135. The method in accordance with claim 133 wherein said at least one autoantibody for a neuronal tissue marker is selected from the group consisting of autoantibodies for GFAP, NSE, S100β, CNPase and fragments thereof.

136. The method in accordance with claim 133 wherein said sample of a bodily fluid is selected from the group consisting of blood, blood products, urine, saliva cerebrospinal fluid and lymph.

137. The method in accordance with claim 126 wherein said at least one additional neuronal tissue marker is selected from the group consisting of GFAP, NSE, S100β, CNPase and fragments thereof and wherein said at least autoantibody for a neuronal tissue marker is selected from the group consisting of autoantibodies for GFAP, NSE, S100β, CNPase and fragments thereof.

138. The method in accordance with claim 137 wherein said sample of a bodily fluid is selected from the group consisting of blood, blood products, urine, saliva cerebrospinal fluid and lymph.

139. The method in accordance with claim 129 wherein said at least one neuronal tissue marker is selected from the group consisting of GFAP, NSE, S100β, CNPase and fragments thereof and wherein at least one autoantibody for a neuronal tissue marker is selected from the group consisting of autoantibodies for GFAP, NSE, S100β, CNPase and fragments thereof.

140. The method in accordance with claim 139 wherein said sample of a bodily fluid is selected from the group consisting of blood, blood products, urine, saliva cerebrospinal fluid and lymph.

141. The method in accordance with claim 133 wherein said at least one additional neuronal tissue marker is selected from the group consisting of GFAP, NSE, S100β, CNPase and fragments thereof and said at least one additional autoantibody for a neuronal tissue marker is selected from the group consisting of autoantibodies for GFAP, NSE, S100β, CNPase and fragments thereof.

142. The method in accordance with claim 141 wherein said sample of a bodily fluid is selected from the group consisting of blood, blood products, urine, saliva cerebrospinal fluid and lymph.

143. The method in accordance with claim 126 wherein said at least autoantibody for a neuronal tissue marker is selected from the group consisting of autoantibodies for GFAP, NSE, S100β, CNPase and fragments thereof.

144. A method in accordance with claim 15 wherein said at-risk population is a target population.
